(19) United States
(12) Patent Application Publication
(10) Pub. No.: US 2024/0042422 A1
(43) Pub. Date: Feb. 8, 2024

(54) METHOD FOR PERFORMING A CONDENSATION REACTION USING A SURFACE-REACTED CALCIUM CARBONATE CATALYST

(71) Applicant: Omya International AG, Oftringen (CH)

(72) Inventors: Jamal FTOUNI, Zofingen (CH); Jose THARUN, Utrecht (NL); Pieter C.A. BRUIJNINCX, Utrecht (NL)

(73) Assignee: Omya International AG, Oftringen (CH)

(21) Appl. No.: 18/245,034

(22) PCT Filed: Sep. 29, 2021
(86) PCT No.: PCT/EP2021/076823

§ 371 Date: Mar. 13, 2023

(30) Foreign Application Priority Data
Oct. 1, 2020 (EP) 20199667.5

(51) Int. Cl. B01J 27/232; C07C 45/45; B01J 35/10

(52) U.S. Cl. CPC B01J 27/232; C07C 45/45; B01J 35/1014; B01J 35/1019

(57) ABSTRACT

The present invention relates to a method for performing a condensation reaction by heterogeneous catalysis using a surface-reacted calcium carbonate catalyst and the use of a dry surface-reacted calcium carbonate as a catalyst. The condensation reaction involves reacting a first substrate comprising a C═O double bond and a second substrate comprising an activated hydrogen to obtain a reaction mixture comprising one or more condensation products and one or more condensation byproducts.

The present invention relates to a method for performing a condensation reaction by heterogeneous catalysis using a surface-reacted calcium carbonate catalyst and the use of a dry surface-reacted calcium carbonate as a catalyst.

Condensation reactions are reactions of organic molecules taking place under the formation of a small molecule, such as water. Therefore, condensation reactions typically show a high atom economy. Furthermore, condensation reactions allow for building carbon-carbon bonds in a controlled and reliable way, making them attractive for the synthesis of diverse molecules in laboratory scale and large-scale industrial applications. As an illustrative example, the aldol condensation reaction of butyraldehyde yields 2-ethylhexenal, which is an important intermediate for several downstream products that can be used as disinfectants, insecticides, solvents, wetting agents, pharmaceuticals, fragrances, additives for plastics, and others. Similarly, the Claisen-Schmidt condensation allows access to α,β-unsaturated carbonyl compounds. The Knoevenagel condensation is an important reaction, which, in combination with an optional saponification and decarboxylation step, allows access to unsaturated compounds, such as α,β-unsaturated carbonyl compounds. Industrially important examples of α,β-unsaturated carbonyl compounds accessible by a Knoevenagel condensation step include sorbic acid and cinnamic acid. Similarly, the Henry reaction allows for the condensation of nitroalkanes with carbonyl compounds to yield nitroalkenes.

Performing a condensation reaction usually requires an acidic or basic catalyst. Common acidic or basic catalysts, such as sodium hydroxide or sulfuric acid, are dissolved in the reaction mixture, which complicates or prevents catalyst recovery and negatively affects the purity of the final product. Alternatively, cyanide-based catalysts have been employed, which are, however, undesirable due to their high toxicity. Therefore, compounds such as alkali metal containing zeolites, alkali earth metal oxides, molybdenum disulfide, amino-functionalized chitosan, niobium(V) oxide, silica on alumina or magnesia on zirconia have been suggested as catalysts in condensation reactions. Alternatively, hydroxyapatites, magnesium oxide, hydrotalcites, bentonite or titanium dioxide have been suggested as catalysts in condensation reactions.

Application WO2019180012 A9 discloses a calcined surface-reacted calcium carbonate as a catalyst for the transesterification of carboxylic acid esters. Therein, it is shown that, for the reaction of an ester and an alcohol in a transesterification reaction, at least partial calcination of the calcium carbonate to calcium oxide is required to achieve catalytic activity.

EP3176222 refers to a method for the production of granules comprising surface-reacted calcium carbonate. The granules can be used in catalytic systems. However, the obtained granules comprising surface-reacted calcium carbonate have a volume median particle size of from 0.1 to 6 mm and, therefore, are much bigger than normal surface-reacted calcium carbonate.

US2016228859 refers to a method for the implementation of organic synthesis reactions, comprising catalysis involving a catalyst of materials of organic origin containing alkali or alkaline-earth metals, and practically devoid of transition metals, metalloids and post-transition metals. The materials of organic origin are marine shells or the shells of non-marine molluscs, said shells containing a significant level of an alkali or alkaline-earth metal, preferably calcium (Ca), preferably in the form of calcium carbonate, in a quantity preferably greater than 80%, more preferentially greater than 90% by weight, said shells having optionally been subjected to grinding and/or a heat treatment. US2016228859 does not refer to surface-reacted calcium carbonate.

Patent application WO2013087211 A1 relates to a method for catalytically condensing organic compounds containing at least one oxo and/or hydroxyl function into CH acidic compounds and/or coupling said organic compounds to the CH acidic compounds in the presence of a catalyst, which comprises an active carbon substrate provided with a metal.

Application WO2008113563 A1 relates to processes for producing ketones, and more specifically, to processes for producing unsaturated ketones using Ca/Na oxide on silica as solid base supported catalysts.

However, these materials are often costly and their production in part is limited to laboratory-scale amounts. Furthermore, some of these materials are less catalytically active due to their low surface area and their surface chemistry and composition. Thus, such materials have to be added in large amounts to the reaction mixture to enable sufficient turnover.

Thus, there is a continued need for further catalyst improvement, i.e., for developing catalysts that are highly catalytically active in a range of condensation type reactions, that can be easily removed from the reaction mixture and reused, and that can be produced at a large scale at reasonable cost. Preferably, the condensation reaction can be performed at mild reaction conditions and with few side reactions, wherein the product is obtained in high yields and can be easily separated from the reaction mixture.

Additionally, the catalyst should be easy to handle and stable upon storage and should be environmentally friendly and non-toxic.

The foregoing and other problems can be solved by the subject-matter as defined herein in the independent claims.

Therefore, a first aspect of the present invention relates to a method for performing a condensation reaction using a surface-reacted calcium carbonate catalyst. The method comprises the steps of
a) providing a first substrate comprising a C═O double bond;
b) providing a second substrate comprising an activated hydrogen;
c) providing a surface-reacted calcium carbonate,
wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 10 m²/g, measured using nitrogen and the BET method according to ISO 9277:2010;
d) activating the surface-reacted calcium carbonate of step c) at a temperature in the range from 100 to 500° C. to obtain a dry surface-reacted calcium carbonate;
e) reacting the first substrate of step a) and the second substrate of step b) in the presence of the dry surface-reacted calcium carbonate of step d) to obtain a reaction mixture comprising a condensation product and one or more condensation byproducts.

The inventors found that a surface-reacted calcium carbonate is a highly active catalyst for condensation type reactions. As will be explained in more detail hereinbelow, surface-reacted calcium carbonate is a specific type of calcium carbonate obtained by reacting a calcium carbonate-containing material, carbon dioxide and one or more H₃O⁺ ion donors. The surface-reacted calcium carbonate has a high specific surface-area and, due to it being surface-reacted, comprises catalytically active acidic and basic sites. Furthermore, the inventors found that activating the surface-reacted calcium carbonate by heating greatly enhances its catalytic activity. It is believed that the activation step ensures to remove essentially all water and volatiles, such as volatile organic molecules, which may have been adsorbed onto the surface-reacted calcium carbonate during storage, from its surface. However, it is to be understood that the composition or surface structure of the surface-reacted calcium carbonate is not altered by the activation step, since the activation takes place below the calcination temperature of the surface-reacted calcium carbonate.

Another aspect of the present invention relates to the use of a dry surface-reacted calcium carbonate as a catalyst, wherein the surface-reacted calcium carbonate is a reaction product of ground natural calcium carbonate-containing mineral (GNCC) or precipitated calcium carbonate (PCC) with carbon dioxide and one or more H₃O+ ion donors and wherein the carbon dioxide is formed in situ by the H₃O⁺ ion donors treatment and/or is supplied from an external source, and wherein the surface-reacted calcium carbonate has a specific surface area of at least 10 m²/g, measured using nitrogen and the BET method according to ISO 9277:2010, and wherein the surface-reacted calcium carbonate has been dried by heating at a temperature in the range from 100 to 500° C.

Advantageous embodiments are defined in the corresponding dependent claims.

In one embodiment of any one of the aspects of the present invention, the first substrate is a compound according to formula (1)

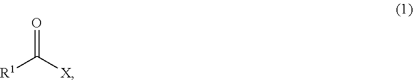

wherein R¹ is selected from the group consisting of
i) a hydrogen atom, and
ii) an organyl group R¹¹, preferably being a linear or branched, saturated or unsaturated, cyclic or acyclic group comprising from 1 to 30 carbon atoms, which is optionally further substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group;
and wherein X is selected from the group consisting of
i) a hydrogen atom,
ii) an organyl group R^{X}, preferably being a linear or branched, saturated or unsaturated, cyclic or acyclic group comprising from 1 to 30 carbon atoms, which is optionally further substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group, and
iii) a leaving group LG, preferably selected from the group consisting of a halide group, an acyloxy group, a sulfate group and a sulfite group,
preferably wherein X is a hydrogen atom.

In another embodiment of any one of the aspects of the present invention, the second substrate is a compound according to formula (2)

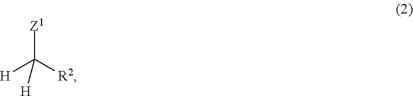

wherein Z¹ is an electron-withdrawing group, preferably selected from the group consisting of an acyl group, a formyl group, an acetyl group, a nitro group, a nitrile group, an ester group, a carboxyl group, a sulfonate group, a sulfinate group, a sulfonyl group, a sulfinyl group and an isocyanate group,
and wherein R² is selected from the group consisting of
i) a hydrogen atom,
ii) an organyl group R²¹, preferably being a linear or branched, saturated or unsaturated, cyclic or acyclic group comprising from 1 to 30 carbon atoms, which is optionally further substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group, and
iii) an electron-withdrawing group Z², preferably selected from the group consisting of an acyl group, a formyl group, an acetyl group, a nitro group, a nitrile group, an ester group, a carboxyl group, a sulfonate group, a sulfinate group, a sulfonyl group, a sulfinyl group and an isocyanate group,
with the proviso that, if Z¹ is an electron-withdrawing group other than an acyl group, a formyl group, an acetyl group or a nitro group, R² is an electron-withdrawing group Z².

In yet another embodiment of any one of the aspects of the present invention, the first substrate is a compound according to formula (1) and the second substrate is a compound according to formula (2), wherein
R¹ is a hydrogen atom or an organyl group R¹¹,
X is a hydrogen atom,
R² is a hydrogen atom or an organyl group R²¹, and
Z¹ is an electron-withdrawing group selected from the group consisting of an acyl group, a formyl group, an acetyl group and a nitro group.

In still another embodiment of any one of the aspects of the present invention, the first substrate is a compound according to formula (1) and the second substrate is a compound according to formula (2), wherein
R¹ is a hydrogen atom or an organyl group R¹¹,
X is a hydrogen atom,
R² is an electron-withdrawing group Z²,
and wherein Z¹ and Z² are independently from each other selected from the group consisting of an acyl group, a formyl group, a nitro group, a nitrile group, and an ester group, preferably Z¹ and Z² are the same group.

In one embodiment of any one of the aspects of the present invention, the first substrate and the second substrate are the same compound.

In another embodiment of any one of the aspects of the present invention, the surface-reacted calcium carbonate of step c) has
i) a volume median particle size (d₅₀) from 0.5 to 50 μm, preferably from 1 to 30 μm, more preferably from 1.5 to 20 μm, and most preferably from 2 to 12 μm, and/or
ii) a top cut (d₉₈) value from 1 to 120 μm, preferably from 2 to 100 μm, more preferably from 5 to 50 μm, and most preferably from 8 to 25 μm, and/or
iii) a specific surface area (BET) from 10 to 200 m²/g, preferably of from 20 to 180 m²/g, more preferably from 25 to 160 m²/g, and most preferably from 30 to 140 m²/g, as measured by the BET method.

In yet another embodiment of any one of the aspects of the present invention, the dry surface-reacted calcium carbonate of step d) has
i) a residual total moisture content from 0.01 wt.-% to 0.75 wt.-%, preferably from 0.02 wt.-% to 0.5 wt.-%, based on the total dry weight of the surface-reacted calcium carbonate, and/or
ii) a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia, and/or
iii) a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide.

In still another embodiment of any one of the aspects of the present invention, the one or more H₃O⁺ ion donors are selected from the group consisting of hydrochloric acid, sulfuric acid, sulfurous acid, phosphoric acid, citric acid, oxalic acid, an acidic salt, acetic acid, formic acid, and mixtures thereof, preferably selected from the group consisting of hydrochloric acid, sulfuric acid, sulfurous acid, phosphoric acid, oxalic acid, H₂PO₄—, being at least partially neutralized by a corresponding cation such as Li⁺, Na⁺or K⁺, HPO₄²⁻, being at least partially neutralized by a corresponding cation such as Li⁺, Na⁺, K⁺, Mg²⁺, or Ca²⁺ and mixtures thereof, more preferably selected from the group consisting of hydrochloric acid, sulfuric acid, sulfurous acid, phosphoric acid, oxalic acid, or mixtures thereof, and most preferably, the one or more H₃O⁺ ion donor is phosphoric acid.

In one embodiment of the process of the present invention, activation step d) is performed at a temperature from 150° C. to 400° C., more preferably from 150° C. to 300° C., and/or for a duration of at least 0.5 h, preferably of at least 1 h, and more preferably of at least 2 h, optionally at a pressure of less than 101.3 kPa.

In another embodiment of the process of the present invention, reaction step e) is performed
i) in the absence of a solvent or in the presence of a solvent, preferably selected from the group comprising acetonitrile, benzene, 1-butanol, 2-butanol, tert-butanol, chlorobenzene, chloroform, cyclohexane, 1,2-dichloroethane, diethylene glycol dimethyl ether, 1,2-dimethoxyethane, dimethyl carbonate, dimethyl formamide, dimethyl sulfoxide, 1,4-dioxane, ethanol, ethyl acetate, ethylene glycol, methanol, methyl tert-butyl ether, cyclopropyl methyl ether, N-methyl pyrrolidinone, 1-propanol, 2-propanol, propylene carbonate, tetrahydrofuran, 2-methyltetrahydrofuran, toluene, xylene, mesitylene and mixtures thereof, preferably in the absence of a solvent, and/or
ii) in the liquid phase at a reaction temperature in the range from 20° C. to 250° C., preferably from 50° C. to 200° C., more preferably from 100 to 150° C.

In yet another embodiment of the process of the present invention, in reaction step e),
i) the dry surface-reacted calcium carbonate is added in an amount from 0.5 to 50 wt.-%, preferably from 1 to 30 wt.-%, more preferably from 5 to 25 wt.-%, and most preferably from 10 to 20 wt.-%, based on the total weight of the first substrate, and/or
ii) the first substrate and the second substrate are added in a molar ratio from 1:1 to 1:20, preferably from 1:2.5 to 1:15, and more preferably from 1:5 to 1:15.

It should be understood that for the purposes of the present invention, the following terms have the following meanings.

A “condensation reaction”, as defined by the IUPAC, refers to a reaction in which two molecules or remote reactive sites within the same molecule yield a main product with accompanying formation of water or of some other small molecule, e.g. ammonia, methanol, ethanol, acetic acid or hydrogen sulfide (see IUPAC Gold Book, https://doi.org/10.1351/goldbook.C01238). The organic main reaction product of said reaction is referred to as “condensation product”, whereas the small molecule is referred to as the “condensation byproduct”. It is to be understood that a “transesterification reaction” does not represent a condensation reaction in the sense of the present invention. A “transesterification reaction” is the process of exchanging the organyl group of an ester with the organyl group R′ of an alcohol.

“Heterogeneous catalysis” is understood to be a catalytic reaction, wherein the reaction occurs at or near an interface between phases, i.e., between the liquid phase comprising the substrates and the solid phase being the heterogeneous catalyst (see IUPAC Gold Book, https://doi.org/10.1351/goldbook.000876).

A “substrate” in the meaning of the present invention refers to an organic compound, which is used as the starting material in the condensation reaction, i.e., is transformed under the influence of the catalyst. It is understood that in the present invention the first and the second substrate may react with each other to form the condensation products and the condensation byproducts.

Thus, it is understood that a “substrate comprising a C═O double bond” is an organic compound comprising, e.g., a carbonyl group, a carboxyl group or an ester group. Carbon dioxide does not represent a substrate comprising a C═O double bond in the sense of the present invention.

Consequently, a “substrate comprising an activated hydrogen” is understood to be an organic compound comprising an “activated hydrogen”, that is, a hydrogen atom in said organic compound, which is bound to a carbon atom bearing an electron-withdrawing group. An “electron-withdrawing group”, in accordance with the understanding of the skilled person, is defined as a group, which draws electrons away from said carbon atom, i.e., increases the partial positive charge on said carbon atom, compared to the same molecule, wherein the electron-withdrawing group is replaced by a hydrogen atom.

An “organyl group” in the meaning of the present invention is any organic substituent group, regardless of functional type, having one free valence at a carbon atom, e.g. CH₃CH₂—, ClCH₂⁻, CH₃C(═O)—, 4-pyridylmethyl—(see IUPAC Gold Book, https://doi.org/10.1351/goldbook.O04329).

A “leaving group” in the meaning of the present invention is an atom or group (charged or uncharged) that becomes detached from an atom in what is considered to be the residual part of the substrate in the condensation reaction (see IUPAC Gold Book, https://doi.org/10.1351/goldbook.L03493). More specifically, the leaving group of the compound according to formula (1) is displaced, i.e., becomes detached, e.g., by reaction with the second substrate. Preferably, the leaving group is an electron-withdrawing group.

A “surface-reacted calcium carbonate” according to the present invention is a reaction product of ground natural calcium carbonate (GNCC) or precipitated calcium carbonate (PCC) treated with carbon dioxide and one or more H₃O⁺ ion donors, wherein the carbon dioxide is formed in situ by the H₃O⁺ ion donors treatment. An H₃O⁺ ion donor in the context of the present invention is a Brønsted acid and/or an acid salt.

Throughout the present document, the term “specific surface area” (“SSA”, in m²/g), which is used to define functionalized calcium carbonate or other materials, refers to the specific surface area as determined by using the BET method (using nitrogen as adsorbing gas), according to ISO 9277:2010.

The “particle size” of surface-reacted calcium carbonate herein is described as volume-based particle size distribution dₓ(vol). Therein, the value dₓ(vol) represents the diameter relative to which x % by volume of the particles have diameters less than dₓ(vol). This means that, for example, the d₂₀(vol) value is the particle size at which 20 vol. % of all particles are smaller than that particle size. The d₅₀(vol) value is thus the volume median particle size, i.e. 50 vol. % of all particles are smaller than that particle size and the des(vol) value, referred to as volume-based top cut, is the particle size at which 98 vol. % of all particles are smaller than that particle size.

Volume median particle size d₅₀ was evaluated using a Malvern Mastersizer 3000 Laser Diffraction System. The d₅₀ or des value, measured using a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

For the purpose of the present invention, the “porosity” or “pore volume” refers to the intra-particle intruded specific pore volume.

In the context of the present invention, the term “pore” is to be understood as describing the space that is found between and/or within particles, i.e. that is formed by the particles as they pack together under nearest neighbor contact (interparticle pores), such as in a powder or a compact, and/or the void space within porous particles (intraparticle pores), and that allows the passage of liquids under pressure when saturated by the liquid and/or supports absorption of surface wetting liquids.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm. The equilibration time used at each pressure step is 20 s. The sample material is sealed in a 3 cm³ chamber powder penetrometer for analysis. The data are corrected for mercury compression, penetrometer expansion and sample material elastic compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 1996, 35(5), 1753-1764).

The total pore volume seen in the cumulative intrusion data is separated into two regions with the intrusion data from 214 μm down to about 1 to 4 μm showing the coarse packing of the sample between any agglomerate structures contributing strongly. Below these diameters lies the fine interparticle packing of the particles themselves. If they also have intraparticle pores, then this region appears bimodal, and by taking the specific pore volume intruded by mercury into pores finer than the modal turning point, i.e. finer than the bimodal point of inflection, we thus define the specific intraparticle pore volume. The sum of these three regions gives the total overall pore volume of the powder, but depends strongly on the original sample compaction/settling of the powder at the coarse pore end of the distribution.

By taking the first derivative of the cumulative intrusion curve, the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range, it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

A “dry” material (e.g., dry surface-reacted calcium carbonate) in the meaning of the present invention has a total or residual moisture content which, unless specified otherwise, is less than or equal to 5.0 wt. %, preferably less than or equal to 0.75 wt. %, more preferably less than or equal to 0.5 wt. %, even more preferably less than or equal to 0.2 wt. %, and most preferably between 0.02 and 0.07 wt. %, based on the total weight of the dried material.

The term “calcining” in the meaning of the present document refers to a thermal treatment process applied to solid materials causing loss of moisture, reduction or oxidation, and additionally the decomposition of carbonates and other compounds resulting in an oxide of the corresponding solid material. It is to be understood that the surface-reacted calcium carbonate of the present invention is not calcined prior to, during and after the activation step d).

The “total number of basic sites” is a measure of the basicity of a solid material and is represented by the total molar amount of carbon dioxide that can be adsorbed on the basic sites of a certain amount of the solid material, and is determined by temperature-programmed desorption with carbon dioxide as described herein.

The “total number of acidic sites” is a measure of the acidity of a solid material and is represented by the total molar amount of ammonia that can be adsorbed on the acidic sites of a certain amount of the solid material, and is determined by temperature-programmed desorption with ammonia as described herein.

“Temperature-programmed desorption”, or thermal desorption spectroscopy, in the meaning of the present invention refers to an analytical method well-known to the skilled person and involves adsorbing a gas, such as carbon dioxide or ammonia, on a sample, subsequently heating said sample and measuring the amount of released gas during the heating process.

Where the term “comprising” is used in the present description and claims, it does not exclude other non-specified elements of major or minor functional importance. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Whenever the terms “including” or “having” are used, these terms are meant to be equivalent to “comprising” as defined above.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This e.g. means that, unless the context clearly dictates otherwise, the term “obtained” does not mean to indicate that, e.g., an embodiment must be obtained by, e.g., the sequence of steps following the term “obtained” even though such a limited understanding is always included by the terms “obtained” or “defined” as a preferred embodiment.

According to one embodiment of the present invention, a method for performing a condensation reaction by heterogeneous catalysis is disclosed. The method comprises the steps of
a) providing a first substrate comprising a C═O double bond;
b) providing a second substrate comprising an activated hydrogen;
c) providing a surface-reacted calcium carbonate,
wherein the surface-reacted calcium carbonate is a reaction product of ground natural calcium carbonate-containing mineral (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source,
and wherein the surface-reacted calcium carbonate has a specific surface area of at least 10 m²/g, measured using nitrogen and the BET method according to ISO 9277:2010;
d) activating the surface-reacted calcium carbonate of step c) at a temperature in the range from 100 to 500° C. to obtain a dry surface-reacted calcium carbonate;
e) reacting the first substrate of step a) and the second substrate of step b) in the presence of the dry surface-reacted calcium carbonate of step d) to obtain a reaction mixture comprising one or more condensation products and one or more condensation byproducts. In the following the steps of the inventive method are described in more detail.

When in the following reference is made to embodiments or technical details of the inventive method for performing a condensation reaction, it is to be understood that these embodiments or technical details also refer to the inventive use of a dry surface-reacted calcium carbonate as a catalyst, as far as applicable.

### Step a)—the First Substrate

According to process step a), a first substrate comprising a C═O double bond is provided. The first substrate is an organic compound, and preferably comprises a carbonyl group. Therefore, it is to be understood that carbon dioxide does not represent a first substrate comprising a C═O double bond in the sense of the present invention. The carbon atom of the C═O group of the first substrate is positively polarized due to the electron-withdrawing effect of the oxygen atom, and therefore, is susceptible to nucleophilic attack. Said susceptibility to nucleophilic attack may be enhanced by interaction of the C═O oxygen atom with the acidic sites of the catalyst.

In a preferred embodiment of the present invention, the first substrate is a compound according to formula (1)

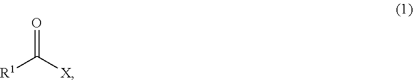

wherein R¹ is selected from the group consisting of
   i) a hydrogen atom, and
   ii) an organyl group R¹¹;
and wherein X is selected from the group consisting of
   i) a hydrogen atom,
   ii) an organyl group R^{X}; and
   iii) a leaving group LG.

There are no particular restrictions as to the organyl groups R¹¹ and R^{X}, as long as they do not prevent the nucleophile from attacking the C═O carbon atom, e.g., by electronic or steric effects. However, in a preferred embodiment, R¹¹ and R^{X} independently from another represent a linear or branched, saturated or unsaturated, cyclic or acyclic group comprising from 1 to 30 carbon atoms, which is optionally further substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group. For example, R¹¹ and/or R^{X} may represent an aryl group comprising from 6 to 30 carbon atoms.

A linear group is understood to be a group, wherein each carbon atom has a direct bond to 1 or 2 other carbon atoms.

A branched group is understood to be a group, wherein at least one carbon atom has a direct bond to 3 or 4 other carbon atoms.

A saturated group is understood to be a group, which does not contain a carbon-carbon multiple bond, i.e., a carbon-carbon double bond or a carbon-carbon triple bond.

An unsaturated group is understood to be a group, which contains at least one carbon-carbon multiple bond, i.e., a carbon-carbon double bond or a carbon-carbon triple bond.

A cyclic group is understood to be a group, wherein at least three carbon atoms are linked together in a way such as to form a ring.

An acyclic group is understood to be a group, wherein no ring is present.

A halide group in the meaning of the present invention is fluoride, chloride, bromide and iodide.

The hydroxy group in the meaning of the present invention is the functional group —OH.

The oxo group in the meaning of the present invention is the functional group ═O.

The alkyl group in the meaning of the present invention refers to a linear or branched, saturated organic compound composed of carbon and hydrogen having 1 to 28, preferably 8 to 26, more preferably 14 to 22, and most preferably 16 to 20 carbon atoms.

The vinyl group in the meaning of the present invention is the functional group —CH═CH₂.

The acyloxy group according to the present invention is an acyl group that is singular bonded to an oxygen atom. The acyl group according to the present invention is an organyl group that is attached to a CO group with a single bond. Thus, the acyloxy group has the formula —O—C(═O)—R^{A1} wherein R^{A1} is an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

According to the present invention, the acyloxy group may be an acryloxy group having the formula —O—C(═O)—C(—R^{A2})═CH₂ wherein R^{A2} is an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

The carboxyl group according to the present invention consists of a carbon atom that forms two chemical bonds to one oxygen atom and one chemical bond to a second oxygen atom. This second oxygen is also bonded to a hydrogen atom. The arrangement is written —C(═O)—OH.

The epoxy group according to the present invention consists of an oxygen atom joined by single bonds to two adjacent carbon atoms, thus forming the three-membered epoxide ring.

An anhydride group comprises two acyl groups bonded to one oxygen atom. According to the present invention, the anhydride group has the chemical formula —C(═O)—O—C(═O)—R^{A3}, wherein R^{A3} is an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group. According to another embodiment the anhydride group is a cyclic anhydride group.

The ester group according to the present invention has the chemical formula —C(═O)—O—R^{A4}wherein R^{A4} is an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

The aldehyde group in the meaning of the present invention is the functional group —C(═O)—H.

The amino group in the meaning of the present invention is the functional group —NH₂, wherein optionally one or both of the hydrogen atoms are replaced by one or two organyl groups selected independently from each other, preferably as described herein within context of the organyl group R¹¹, and more preferably are alkyl groups, preferably comprising from 1 to 18 carbon atoms, e.g., from 2 to 12 carbon atoms, such as a methyl group or an ethyl group.

The ureido group in the meaning of the present invention is the functional group —NH—C(═O)—NH₂.

The azide group in the meaning of the present invention is the functional group —N₃.

The phosphonate group according to the present invention has the chemical formula —P(═O)(OR^{A5})(OR^{A6}), wherein R^{A5} and R^{A6} are independently from each other selected from the group consisting of hydrogen and an organyl group preferably as described herein within context of the organyl group R¹¹, and more preferably are alkyl groups, preferably comprising from 1 to 18 carbon atoms, e.g., from 2 to 12 carbon atoms, such as a methyl group or an ethyl group.

The phosphine group according to the present invention has the chemical formula —PR^{A7}R^{A8}, wherein R^{A7} and R^{A8} are independently from each other selected from the group consisting of hydrogen and an organyl group preferably as described herein within context of the organyl group R¹¹, and more preferably are alkyl groups, preferably comprising from 1 to 18 carbon atoms, e.g., from 2 to 12 carbon atoms, such as a methyl group or an ethyl group.

The sulfonate group in the meaning of the present invention is the functional group —S(═O)(═O)—OR^{A9}, wherein R^{A9} is selected from the group consisting of hydrogen and an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

The sulfide group according to the present invention has the chemical formula —SR^{A10} wherein R^{A10} is a hydrogen or an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

The disulfide group according to the present invention has the chemical formula —S—S—R^{A11}wherein R^{A11} is a hydrogen or an organyl group preferably as described herein within context of the organyl group R¹¹, and more preferably is an alkyl group, preferably comprising from 1 to 18 carbon atoms, e.g., from 2 to 12 carbon atoms, such as a methyl group or an ethyl group.

The isocyanate group in the meaning of the present invention is the functional group —N═C(═O). A masked isocyanate group according to the present invention refers to an isocyanate group that is masked or blocked by a masking agent. At temperatures of above 120° C., the masking agent will be split off and the isocyanate group will be obtained.

The thiol group in the meaning of the present invention is the functional group —SH.

The phenyl group or phenyl ring in the meaning of the present invention is a cyclic group with the formula —C₆H₅.

The benzyl group in the meaning of the present invention is the functional group —CH₂C₆H₅.

The styryl group in the meaning of the present invention is the functional group —CH═CH—C₆H₅.

The benzoyl group in the meaning of the present invention is the functional group —C(═O)C₆H₅.

The sulfinate group in the meaning of the present invention is the functional group —S(═O)—OR^{A12}, wherein R^{A12} is selected from the group consisting of hydrogen and an organyl group preferably as described herein within context of the organyl group R¹¹.

The sulfonyl group in the meaning of the present invention is the functional group —S(═O)₂—R^{A13} wherein R^{A13} is selected from the group consisting of hydrogen and an organyl group preferably as described herein within context of the organyl group R¹¹.

The sulfinyl group in the meaning of the present invention is the functional group —S(═O)—R^{A14}wherein R^{A14} is selected from the group consisting of hydrogen and an organyl group preferably as described herein within context of the organyl group R¹¹.

The aryl group in the meaning of the present invention is a phenyl group, which is optionally further substituted with one or more organyl groups and/or one or more functional groups, e.g., as described hereinabove.

In a preferred embodiment of the present invention, the first substrate is a compound according to formula (1), wherein X is a hydrogen atom (X═H) and R¹ is an organyl group R¹¹ (R¹═R¹¹). In this embodiment, compound (1) is an aldehyde. An “aldehyde” in the meaning of the present invention is a compound RC(═O)H, in which a carbonyl group is bonded via the carbon atom to one hydrogen atom and to one organyl group R.

The first substrate being a compound according to formula (1), wherein X═H and R¹═R¹¹ comprises an organyl group R¹¹ being a linear or branched, saturated or unsaturated, cyclic or acyclic group comprising from 1 to 30 carbon atoms, preferably from 1 to 20 carbon atoms, more preferably from 1 to 15 carbon atoms, which is optionally further substituted by one or more groups selected from the group consisting of a halide group, a hydroxy group, an oxo group, an alkyl group, a vinyl group, an alkoxy group, an acyloxy group, a carbonyl group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

The linear or branched, saturated or unsaturated, cyclic or acyclic group may be selected from the group consisting of an alkyl group, an alkenyl group, a cycloalkyl group, a cycloalkenyl group, an aryl group and a heteroaryl group, wherein each of the alkyl group, the alkenyl group, the cycloalkyl group, the cycloalkenyl group and the aryl group may be optionally further substituted by one or more groups selected from the group consisting of a halide group, a hydroxy group, an oxo group, an alkyl group, a vinyl group, an alkoxy group, an acyloxy group, a carbonyl group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

Exemplary organyl groups R¹¹ being alkyl groups, which are optionally further substituted, include methyl, ethyl, propyl, butyl, isobutyl, nonyl, chloromethyl, trichloromethyl, benzyl, formyl, acetyl, and carboxyl groups. The corresponding compounds according to formula (1) are acetaldehyde, propionic aldehyde, butyraldehyde, isobutyraldehyde, nonanal, chloroacetaldehyde, trichloroacetaldehyde, phenylacetaldehyde, glyoxal and glyoxylic acid, respectively.

Exemplary organyl groups R¹¹ being alkenyl groups, which are optionally further substituted, include vinyl, 1-allyl, 2-methylbut-1-en-1-yl, (all-E)-2,6-dimethyl-9-(2,6,6-trimethylcyclohexen-1-yl)octa-1,3,5,7-tetraen-1-yl, and phenylethenyl groups. The corresponding compounds according to formula (1) are acrolein, crotonic aldehyde, 3-methylbut-2-enal, retinal and cinnamic aldehyde, respectively.

Exemplary organyl groups R¹¹ being cycloalkyl groups, which are optionally further substituted, include cyclopropyl, cyclobutyl, cyclopentyl and cyclohexyl groups. The corresponding compounds according to formula (1) are cyclopropanecarbaldehyde, cyclobutanecarbaldehyde, cyclopentanecarbaldehyde and cyclohexanecarbaldehyde, respectively.

Exemplary organyl groups R¹¹ being aryl groups or heteroaryl groups, which are optionally further substituted, include phenyl, 4-methylphenyl, 4-hydroxy-3-methoxyphenyl, pyridyl, furyl, methylfuryl, hydroxymethylfuryl and thiophenyl groups. The corresponding compounds according to formula (1) are benzaldehyde, 4-methylbenzaldehyde, vanillin, pyridine-2-carboxaldehyde, furfural, 5-methylfuryl, 5-hydroxymethylfurfural and 2-thiophencarbaldehyde, respectively.

In a preferred embodiment of the present invention, the first substrate is a compound according to formula (1), wherein X is a hydrogen atom (X═H) and R¹ is an organyl group R¹¹ (R¹═R¹¹), R¹¹ equals—(CH₂)—R¹², wherein R¹² represents a linear or branched, saturated or unsaturated, cyclic or acyclic group comprising from 1 to 29 carbon atoms, preferably from 1 to 19 carbon atoms, more preferably from 1 to 14 carbon atoms, which is optionally further substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

As will be outlined below, aldehydes being a compound according to formula (1), wherein X═H and R¹═R¹¹ may be used in the aldol condensation reaction. Such compounds may be used in the self-aldol condensation reaction, provided that they comprise both a C═O group and an activated hydrogen. The self-aldol condensation reaction is understood to be an aldol condensation reaction, wherein the first substrate and the second substrate are the same compound. However, such aldehydes may also be used in the cross-aldol condensation reaction. The cross-aldol condensation reaction is understood to be an aldol condensation reaction wherein the first substrate and the second substrate are different compounds. In one specific embodiment, the compound according to formula (1), wherein X═H and R¹═R¹¹, does not comprise a hydrogen atom in the α-position of the carbonyl group, and preferably, R¹¹ is an aryl group. Such compound may be used in the Claisen-Schmidt reaction.

In another preferred embodiment of the present invention, the first substrate is a compound according to formula (1), wherein X is a hydrogen atom (X═H) and R¹ is a hydrogen atom (R¹═H). In said embodiment, the first substrate is formaldehyde (HCHO). As will be outlined below, formaldehyde may be employed as a substrate in a cross-aldol condensation reaction or in a Knoevenagel reaction.

In another embodiment of the present invention, the first substrate is a compound according to formula (1), wherein X is an organyl group R^{X} (X═R^{X}) and R¹ is an organyl group R¹¹ (R¹═R¹¹). In this embodiment, the compound according to formula (1) is a ketone. In the meaning of the present invention, a “ketone” is understood to be a compound in which a carbonyl group is bonded via the carbon atom to two carbon atoms RR′C═O, wherein R and R′ are organyl groups, i.e., neither R nor R′ may be H. Alternatively, R^{X} and R¹ may be adjoined with each other to form a cyclic system.

The first substrate being a compound according to formula (1), wherein X═R^{X} and R¹═R¹, comprises an organyl group R¹¹ being a linear or branched, saturated or unsaturated, cyclic or acyclic group comprising from 1 to 30 carbon atoms, preferably from 1 to 20 carbon atoms, more preferably from 1 to 15 carbon atoms, which is optionally further substituted by one or more groups selected from the group consisting of a halide group, a hydroxy group, an oxo group, an alkyl group, a vinyl group, an alkoxy group, an acyloxy group, a carbonyl group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

The linear or branched, saturated or unsaturated, cyclic or acyclic group may be selected from the group consisting of an alkyl group, an alkenyl group, a cycloalkyl group, a cycloalkenyl group, an aryl group and a heteroaryl group, wherein each of the alkyl group, the alkenyl group, the cycloalkyl group, the cycloalkenyl group and the aryl group may be optionally further substituted as defined above.

The first substrate being a compound according to formula (1), wherein X═R^{X} and R¹═R¹, comprises an organyl group R^{X} being a linear or branched, saturated or unsaturated, cyclic or acyclic group comprising from 1 to 30 carbon atoms, preferably from 1 to 20 carbon atoms, more preferably from 1 to 15 carbon atoms, which is optionally further substituted by one or more groups selected from the group consisting of a halide group, a hydroxy group, an oxo group, an alkyl group, a vinyl group, an alkoxy group, an acyloxy group, a carbonyl group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

The linear or branched, saturated or unsaturated, cyclic or acyclic group may be selected from the group consisting of an alkyl group, an alkenyl group, a cycloalkyl group, a cycloalkenyl group, an aryl group and a heteroaryl group, wherein each of the alkyl group, the alkenyl group, the cycloalkyl group, the cycloalkenyl group and the aryl group may be optionally further substituted as defined above.

In a preferred embodiment of the present invention, the first substrate is a compound according to formula (1), wherein X═R^{X} and R¹═R¹¹, wherein R¹¹═—(CH₂)—R¹², wherein R¹² represents a linear or branched, saturated or unsaturated, cyclic or acyclic group comprising from 1 to 29 carbon atoms, preferably from 1 to 19 carbon atoms, more preferably from 1 to 14 carbon atoms, which is optionally further substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group; and R^{X}═—(CH₂)—R^{X1}, wherein R^{X1} represents a linear or branched, saturated or unsaturated, cyclic or acyclic group comprising from 1 to 29 carbon atoms, preferably from 1 to 19 carbon atoms, more preferably from 1 to 14 carbon atoms, which is optionally further substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group.

Exemplary first substrates according to the present invention are compounds according to formula (1), wherein X═R^{X} and R¹═R¹¹, including acetone, butanone, pentanone, cyclopentanone, cyclohexanone, methyl vinyl ketone, cyclopropyl methyl ketone, isophorone, acetophenone, benzophenone, benzylidene acetone, dibenzylidene acetone, acetylacetone, diacetyl, chloroacetone, acetoin, diacetone alcohol and ethyl acetoacetate.

In another embodiment of the present invention, the first substrate is a compound according to formula (1), wherein X is an organyl group R^{X} (X═R^{X}) and R¹ is a hydrogen atom (R¹═H). The resulting compound according to formula (1) in said embodiment corresponds to a compound according to formula (1), wherein R¹═R¹¹ and X═H, which has already been described above.

In yet another embodiment of the present invention, the first substrate is a compound according to formula (1), wherein X is a leaving group LG and R¹ is selected from the group consisting of a hydrogen atom and an organyl group R¹¹. It is appreciated that the organyl group R¹¹ is as defined hereinabove. The leaving group LG may be selected from the group consisting of a halide group, an acyloxy group, a sulfate group and a sulfite group, and preferably is selected from the group consisting of a halide group and an acyloxy group. Such first substrate may be employed in a Perkin reaction.

Thus, in one embodiment of the present invention, the first substrate is a compound according to formula (1), wherein R═R¹¹ and X=LG, the leaving group preferably is selected from the group consisting of a halide group, an acyloxy group, a sulfate group and a sulfite group, and preferably is selected from the group consisting of a halide group and an acyloxy group. Thus, the first substrate is an acyl halide, a symmetrical or mixed carboxylic acid anhydride, a mixed anhydride of a carboxylic acid and a sulfonic acid, or a mixed anhydride of a carboxylic acid and a sulfinic acid, respectively.

For the purposes of the present invention, a “mixed anhydride” is considered to be an anhydride formed from the hypothetical condensation reaction of two different acid molecules under the extrusion of one molecule of water. Analogously, a “symmetrical anhydride” is considered to be an anhydride formed from the hypothetical condensation reaction of two identical acid molecules under the extrusion of one molecule of water. In the case that LG=acyloxy group, it is preferred that the provided anhydride is a symmetrical anhydride.

Thus, in another embodiment of the present invention, the first substrate is a compound according to formula (1), wherein R═H and X=LG, the leaving group preferably is selected from the group consisting of a halide group, a sulfate group and a sulfite group. Thus, the first substrate is a formyl halide, a mixed anhydride of formic acid and a sulfonic acid or a mixed anhydride of formic acid and a sulfinic acid, respectively.

In any of the embodiments of the present invention, the leaving group LG being a halide group may be selected from the group consisting of chloride, bromide and iodide.

In any of the embodiments of the present invention, the leaving group LG being a sulfate group is a compound according to the formula —O—S(═O)₂—R^{L3}, wherein R^{L3} is an organyl group preferably as described hereinabove within context of the organyl group R¹¹ and more preferably R^{L3}is methyl, trifluoromethyl, phenyl, or 4-methylphenyl.

In any of the embodiments of the present invention, the leaving group LG being a sulfite group is a compound according to the formula —O—S(═O)—R^{L4}, wherein R^{L4} is an organyl group preferably as described hereinabove within context of the organyl group R¹¹ and more preferably R^{L4} is methyl, trifluoromethyl, phenyl, or 4-methylphenyl.

In another preferred embodiment of the present invention, the first substrate and the second substrate are the same compound. That is, said compound comprises both a C═O double bond and an activated hydrogen. Therefore, one molecule of said compound may react with itself in an intramolecular fashion, or one molecule of said compound may react with another molecule of said compound in an intermolecular fashion.

For the purposes of the present invention, the term “intramolecular” refers to a chemical reaction between different parts of the same molecule. Analogously, the term “intermolecular” refers to a chemical reaction between two or more different molecules.

Thus, in one embodiment, wherein the first substrate and the second substrate are the same compound, and wherein the condensation reaction occurs in an intramolecular fashion to form the one or more condensation products, the first substrate and the second substrate is a compound according to the following formula (3),

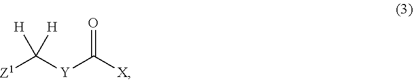

wherein Y represents a tether having from 2 to 7 members, preferably from 3 to 5 members, such that when the substrate is reacted in step e), a 4- to 9-membered ring, preferably a 5- to 7-membered ring is formed in the obtained one or more condensation product, wherein each member may be independently selected from the group consisting of O, NH, NR^{Y1}, CR^{Y2}R^{Y3}, S, C(OR^{Y4})(OR^{Y5}) and the like, wherein R^{Y1} through R^{Y5} represent an organyl group which preferably is an organyl group as described hereinabove within context of the organyl group R¹¹, and wherein Z¹ is as defined above. Preferably, Y is selected from the group consisting of an ethylene group (CH₂—CH₂), a propylene group (CH₂—CH₂—CH₂), an ethyleneoxy group (CH₂CH₂—O), an ethyleneamino group (CH₂CH₂—NR^{Y1}), a butylene group, a propyleneoxy group, a propyleneamino group, a pentylene group, a butyleneoxy group, a CH₂—C(CH₃)₂-CH₂ group and a CH₂—C(OC(═O)R^{Y6})₂-CH₂ group, wherein R^{Y6} represents an organyl group which preferably is an organyl group as described hereinabove within context of the organyl group R¹¹, more preferably methyl or ethyl.

In a preferred embodiment, wherein the first substrate and the second substrate are the same compound, and wherein the condensation reaction occurs in an intramolecular fashion to form the one or more condensation products, the first substrate and the second substrate is a compound according to the following formula (4),

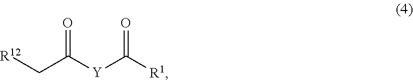

wherein R¹² and R¹ are as defined above and Y represents a tether as defined above. Preferably, R¹²═H, R¹═H or methyl and Y is selected from the group consisting of an ethylene group (CH₂—CH₂), a propylene group (CH₂—CH₂—CH₂), an ethyleneoxy group (CH₂CH₂—O), an ethyleneamino group (CH₂CH₂—NR^{Y1}), a butylene group, a propyleneoxy group, a propyleneamino group, a pentylene group, a butyleneoxy group, a CH₂—C(CH₃)₂-CH₂ group and a CH₂—C(OC(═O)R^{Y6})₂-CH₂ group, wherein R^{Y6} is as defined above. In a particularly preferred embodiment, the compound according to formula (4) is hexane-2,5-dione or heptane-2,6-dione.

In another embodiment, wherein the first substrate and the second substrate are the same compound, and wherein the condensation reaction occurs in an intermolecular fashion, the first substrate and the second substrate preferably is a compound according to formula (1), wherein R¹═—(CH₂)—R¹². It is particularly preferred that the first substrate is a compound according to formula (1), wherein X═H and R¹ represents a linear or branched, acyclic alkyl group. Thus, it is preferred that the first substrate is selected from the group consisting of acetaldehyde, propionic aldehyde, butyric aldehyde, isobutyric aldehyde, n-pentanal, 3-methylbutanal, n-hexanal, 3-methylpentanal, 4-methylpentanal, n-heptanal, 3-methylhexanal, 4-methylhexanal, 5-methylhexanal, n-octanal, 2-ethylhexanal, n-nonanal, n-decanal and n-dodecanal.

It is to be understood that the first substrate is selected in accordance with the second substrate such that, when both substrates are reacted with each other in step e), the desired one or more condensation product(s) is/are formed.

### Step b)—the Second Substrate

According to process step b), a second substrate comprising an activated hydrogen is provided. The second substrate is an organic compound, which comprises said activated hydrogen being attached onto a carbon atom bearing an electron-withdrawing group.

The second substrate can be activated by the interaction with the catalyst. For example, the second substrate can interact with a basic site of the catalyst, and the activated hydrogen can be removed by deprotonation, yielding an anion of the second substrate. However, certain second substrates can also be tautomerized by interaction with the basic and/or acidic sites of the catalyst, yielding, e.g., an enol.

In one embodiment of the present invention, the activated hydrogen of the second substrate has a pKₐ of less than 28, preferably less than 25, more preferably less than 24, and most preferably less than 23, measured in DMSO. The pKₐ represents a measure of the acid strength of the second substrate and corresponds to the (hypothetical) abstraction of the activated hydrogen from the second substrate, yielding a proton and the corresponding anion of the second substrate. The pKₐ value primarily depends on the substituents of the carbon atom bearing the activated hydrogen and can be gathered from standard textbooks and/or tables.

In a preferred embodiment of the present invention, the second substrate comprises two germinal activated hydrogens, i.e., two activated hydrogens bound to the same carbon atom.

In a preferred embodiment of the present invention, the second substrate is a compound according to formula (2)

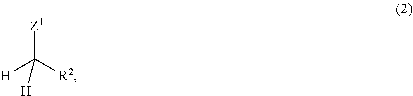

wherein Z¹ is an electron-withdrawing group
and wherein R² is selected from the group consisting of
i) a hydrogen atom,
ii) an organyl group R²¹, and
iii) an electron-withdrawing group Z²,
with the proviso that, if Z¹ is an electron-withdrawing group other than an acyl group, a formyl group, an acetyl group or a nitro group, R² is an electron-withdrawing group Z².

Preferably, Z¹ and Z² are independently from each other selected from the group consisting of an acyl group, a formyl group, an acetyl group, a nitro group, a nitrile group, an ester group, a carboxyl group, a sulfonate group, a sulfinate group, a sulfonyl group, a sulfinyl group and an isocyanate group, and more preferably are independently from each other selected from the group consisting of an acyl group, a formyl group, an ester group and a nitro group.

The formyl group in the meaning of the present invention is the functional group —C(═O)—H.

The nitro group in the meaning of the present invention is the functional group —NO₂.

The nitrile group in the meaning of the present invention is the functional group —CN.

The remaining groups have already been described under step a).

The organyl group R²¹ preferably is a linear or branched, saturated or unsaturated, cyclic or acyclic group comprising from 1 to 30 carbon atoms, which is optionally further substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group. Preferably, the organyl group R²¹ is as described hereinabove within context of the organyl group R¹¹.

In a preferred embodiment of the present invention, the second substrate is a compound according to formula (2), wherein Z¹ is an electron-withdrawing group, selected from the group consisting of an acyl group, a formyl group, an acetyl group and a nitro group, and R² is a hydrogen atom or an organyl group R²¹. In the case where R²═H, the second substrate is a compound according to the formula Z¹—CH₃. Preferably, R²═R²¹.

In the case that Z¹ is an acyl group and R²═R²¹, the second substrate is a ketone having the formula R^{Z1}—C(═O)—CH₂—R²¹, wherein R^{Z1} is an organyl group preferably as described hereinabove within context of the organyl group R¹¹. Alternatively, R^{Z1} and R²¹ may be adjoined with each other to form a cyclic system. Preferably, if R^{Z1} and R²¹ are adjoined with each other, the moiety R^{Z1}—R²¹ is a tether Y, wherein Y is as described above. Exemplary ketones suitable for use as the second substrate include acetone, butanone, cyclopentanone, cyclohexanone, cyclopropyl methyl ketone, acetophenone, benzylidene acetone and butanedione.

Alternatively, if Z¹ is a formyl group, i.e., a —C(═O)—H moiety, the second substrate is an aldehyde having the formula H—C(═O)—CH₂—R². Exemplary aldehydes include acetaldehyde, propionic aldehyde, butyric aldehyde, nonanal and phenylacetaldehyde.

In the case that Z¹ is an acetyl group, the second substrate is a methyl ketone, having the formula CH₃—C(═O)—CH₂—R².

If Z¹ is a nitro group, the second substrate is a compound according to the formula O₂N—CH₂—R², preferably selected from the group comprising nitromethane, nitroethane, nitropropane, nitrobutane, nitropentane, nitrohexane, nitroheptane and nitrooctane, and most preferably selected from the group consisting of nitromethane, nitrobutane, nitropentane and nitrohexane.

If Z¹ is a nitrile group, the second substrate is a compound according to the formula NC—CH₂—Z², such as malononitrile and cyanoacetaldehyde.

In the case that Z¹ is an ester group, the second substrate is a compound according to the formula R^{A4}—O—C(═O)—CH₂—Z².

In the case that Z¹ is a carboxyl group, the second substrate is a carboxylic acid according to the formula HO—C(═O)—CH₂—Z².

In a preferred embodiment of the present invention, the second substrate is a compound according to formula (2), wherein Z¹ is an electron-withdrawing group as defined above and R² is an electron-withdrawing group Z², preferably selected from the consisting of an acyl group, a formyl group, a nitro group, a nitrile group, and an ester group. More preferably, Z¹ and Z² are independently from each other selected from the group consisting of an acyl group, a formyl group, an ester group and a nitrile group. Even more preferably, Z¹ and Z² are the same group, and especially preferably are selected from the group consisting of an acyl group, a formyl group, an ester group and a nitrile group. Exemplary compounds of this embodiment include malonic acid, mono- and diesters thereof, ethyl acetoacetate, methyl acetoacetate, acetyl acetone, and malononitrile. Such compounds may be employed in the Knoevenagel condensation reaction, as will be outlined in detail below.

In another preferred embodiment of the present invention, the second substrate is a compound according to formula (2), wherein Z¹ is a nitro group and R² is a hydrogen atom or an organyl group R²¹. In that embodiment, the second substrate can be employed in the Henry reaction, as will be outlined in detail below.

In another preferred embodiment of the present invention, the first substrate and the second substrate are the same compound. That is, said compound comprises both a C═O double bond and an activated hydrogen. Therefore, one molecule of said compound may react with itself in an intramolecular fashion, or one molecule of said compound may react with another molecule of said compound in an intermolecular fashion. Said embodiment has been described under step a) above.

It is to be understood that the second substrate is selected together with the first substrate such that, when both substrates are reacted with each other in step e), the desired one or more condensation product is formed.

### Step c)—the Surface-Reacted Calcium Carbonate

According to process step c), a surface-reacted calcium carbonate is provided. The surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source. A H₃O⁺ ion donor in the context of the present invention is a Brønsted acid and/or an acid salt.

In a preferred embodiment of the invention, the surface-reacted calcium carbonate is obtained by a process comprising the steps of: (a) providing a suspension of natural or precipitated calcium carbonate, (b) adding at least one acid having a pKₐ value of 0 or less at 20° C. or having a pKₐ value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterized in that: (i) the at least one acid of step B) has a pKₐ of greater than 2.5 and less than or equal to 7 at 20° C., associated with the ionization of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKₐ of greater than 7 at 20° C., associated with the ionization of the first available hydrogen, and the salt anion of which is capable of forming water-insoluble calcium salts, is additionally provided.

“Natural ground calcium carbonate” (GCC) preferably is selected from calcium carbonate containing minerals selected from the group comprising marble, chalk, limestone and mixtures thereof. Natural calcium carbonate may comprise further naturally occurring components such as magnesium carbonate, alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulverizer, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂ and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S—PCC), rhombohedral (R—PCC), hexagonal prismatic, pinacoidal, colloidal (C—PCC), cubic, and prismatic (P—PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

According to one embodiment of the present invention, the natural or precipitated calcium carbonate is in form of particles having a weight median particle size d₅₀ of 0.05 to 10.0 μm, preferably 0.2 to 5.0 μm, more preferably 0.4 to 3.0 μm, most preferably 0.6 to 1.2 μm, especially 0.7 μm. According to a further embodiment of the present invention, the natural or precipitated calcium carbonate is in form of particles having a top cut particle size des of 0.15 to 55 μm, preferably 1 to 40 μm, more preferably 2 to 25 μm, most preferably 3 to 15 μm, especially 4 μm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKₐ of 0 or less at 20° C.

According to another embodiment, the at least one H₃O⁺ ion donor is a medium-strong acid having a pKₐ value from 0 to 2.5 at 20° C. If the pKₐ at 20° C. is 0 or less, the acid is preferably selected from sulfuric acid, hydrochloric acid, or mixtures thereof. If the pKₐ at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO4 or H₂PO₄⁻, being at least partially neutralized by a corresponding cation such as Li⁺, Na⁺or K⁺, or HPO₄²⁻, being at least partially neutralized by a corresponding cation such as Li⁺, Na⁺, K⁺, Mg²⁺or Ca²⁺. The at least one H₃O⁺ ion donor can also be a mixture of one or more acids and one or more acidic salts.

According to still another embodiment, the at least one H₃O⁺ ion donor is a weak acid having a pKₐ value of greater than 2.5 and less than or equal to 7, when measured at 20° C., associated with the ionization of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionization of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H₃O⁺ ion donor is selected from the group consisting of hydrochloric acid, sulfuric acid, sulfurous acid, phosphoric acid, citric acid, oxalic acid, acetic acid, an acidic salt formic acid, and mixtures thereof. Preferably the at least one H₃O⁺ ion donor is selected from the group consisting of hydrochloric acid, sulfuric acid, sulfurous acid, phosphoric acid, oxalic acid, H₂PO₄⁻, being at least partially neutralized by a corresponding cation such as Li⁺, Na⁺or K⁺, HPO₄²⁻, being at least partially neutralized by a corresponding cation such as Li⁺, Na⁺, K⁺, Mg²⁺, or Ca²⁺ and mixtures thereof, more preferably the at least one H₃O⁺ ion donor is selected from the group consisting of hydrochloric acid, sulfuric acid, sulfurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulfuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

Further details about the preparation of the surface-reacted natural calcium carbonate are disclosed in WO0039222 A1, WO2004083316 A1, WO2005121257 A2, WO2009074492 A1, EP2264108 A1, EP2264109 A1 and US20040020410 A1, the content of these references herewith being included in the present application.

Similarly, surface-reacted precipitated calcium carbonate is obtained. As can be taken in detail from WO2009074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H₃O⁺ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H₃O⁺ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H₃O⁺ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO2004083316 A1, the content of this reference herewith being included in the present application.

The surface-reacted calcium carbonate can be kept in suspension, optionally further stabilized by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

In a particularly preferred embodiment of the present invention, the surface-reacted calcium carbonate is a reaction product of natural ground calcium carbonate with carbon dioxide and one or more H₃O⁺ ion donors, wherein the carbon dioxide is formed in situ by the H₃O⁺ ion donors treatment, and wherein the one or more H₃O⁺ ion donor is phosphoric acid. In said embodiment, it is preferred that the surface-reacted calcium carbonate comprises phosphate groups and has an atomic ratio of calcium to phosphorus atoms of at most 3.0, more preferably at most 2.5, and most preferably at most 2.3, determined by XPS.

It is to be understood that the surface-reacted calcium carbonate is not a calcined material.

In a preferred embodiment, the surface-reacted calcium carbonate has a specific surface area of from 10 m²/g to 200 m²/g, preferably from 20 m²/g to 180 m²/g, more preferably from 25 m²/g to 160 m²/g, even more preferably from 30 m²/g to 140 m²/g, most preferably from 50 m²/g to 140 m²/g, measured using nitrogen and the BET method. For example, the surface-reacted calcium carbonate has a specific surface area of from 75 m²/g to 120 m²/g, measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m²/g.

According to a preferred embodiment of the present invention, the surface-reacted calcium carbonate has a specific surface area of from 75 m²/g to 165 m²/g, for example about 160 m²/g, measured using nitrogen and the BET method.

It is furthermore preferred that the surface-reacted calcium carbonate particles have a volume median particle size d₅₀ (vol) of from 0.5 to 50 μm, preferably from 1 to 30 μm, more preferably 1.5 to 20 μm, even more preferably from 2 to 12 μm, and most preferably from 5 to 10 μm.

It may furthermore be preferred that the surface-reacted calcium carbonate particles have a top cut des (vol) value of from 1 to 120 μm, preferably from 2 to 100 μm, more preferably 5 to 50 μm, even more preferably from 8 to 25 μm, and most preferably from 10 to 20 μm.

Therefore, the surface-reacted calcium carbonate particles are really small and differ from granules that normally have a volume median particle size d₅₀ (vol) of at least 0.1 mm.

The value d, represents the diameter relative to which x % of the particles have diameters less than dₓ. This means that the des value is the particle size at which 98% of all particles are smaller. The des value is also designated as “top cut”. The dₓ values may be given in volume or weight percent. The d₅₀ (wt) value is thus the weight median particle size, i.e. 50 wt.-% of all grains are smaller than this particle size, and the d₅₀ (vol) value is the volume median particle size, i.e. 50 vol.-% of all grains are smaller than this particle size.

Volume median grain diameter d₅₀ was evaluated using a Malvern Mastersizer 3000 Laser Diffraction System. The d₅₀ or des value, measured using a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

The weight median grain diameter of the natural ground calcium carbonate and precipitated calcium carbonate is determined by the sedimentation method, which is an analysis of sedimentation behaviour in a gravimetric field. The measurement is made with a Sedigraph™ 5120, Micromeritics Instrument Corporation. The method and the instrument are known to the skilled person and are commonly used to determine grain size of fillers and pigments. The measurement is carried out in an aqueous solution of 0.1 wt.-% Na₄P₂O₇. The samples were dispersed using a high-speed stirrer and sonicated.

The processes and instruments are known to the skilled person and are commonly used to determine grain size of fillers and pigments.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step is 20 seconds. The sample material is sealed in a 5 cm³ chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p1753-1764).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Preferably, the surface-reacted calcium carbonate has an intra-particle intruded specific pore volume in the range from 0.1 to 2.3 cm³/g, more preferably from 0.2 to 2.0 cm³/g, especially preferably from 0.4 to 1.8 cm³/g and most preferably from 0.6 to 1.6 cm³/g, calculated from mercury porosimetry measurement.

The intra-particle pore size of the surface-reacted calcium carbonate preferably is in a range of from 0.004 to 1.6 μm, more preferably in a range of from 0.005 to 1.3 μm, especially preferably from 0.006 to 1.15 μm and most preferably of 0.007 to 1.0 μm, determined by mercury porosimetry measurement.

According to a preferred embodiment of the present invention, the surface-reacted calcium carbonate has a volume median particle size d₅₀ (vol) of from 0.5 to 50 μm, preferably from 1 to 30 μm, more preferably 1.5 to 20 μm, even more preferably from 2 to 12 μm, and most preferably from 5 to 10 μm and has a specific surface area of from 75 m²/g to 165 m²/g, for example about 160 m²/g, measured using nitrogen and the BET method.

According to a preferred embodiment of the present invention, in step c) a surface-reacted calcium carbonate is provided, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, wherein the surface-reacted calcium carbonate has a specific surface area of from 75 m²/g to 165 m²/g, measured using nitrogen and the BET method according to ISO 9277:2010.

According to another preferred embodiment of the present invention, in step c) a surface-reacted calcium carbonate is provided, wherein the surface-reacted calcium carbonate is a reaction product of natural ground calcium carbonate or precipitated calcium carbonate with carbon dioxide and one or more H₃O⁺ ion donors, wherein the carbon dioxide is formed in situ by the H₃O⁺ ion donors treatment and/or is supplied from an external source, wherein the surface-reacted calcium carbonate has a specific surface area of from 75 m²/g to 165 m²/g, measured using nitrogen and the BET method according to ISO 9277:2010 and has a volume median particle size d₅₀ (vol) of from 0.5 to 50 μm, preferably from 1 to 30 μm, more preferably 1.5 to 20 μm, even more preferably from 2 to 12 μm, and most preferably from 5 to 10 μm.

### Step d)—Activation

According to process step d), the surface-reacted calcium carbonate is activated at a temperature in the range from 100 to 500° C. to obtain a dry surface-reacted calcium carbonate. The activation step d) serves to remove impurities such as water and volatile organic compounds, such as alcohols, which may be adsorbed on the surface of the surface-reacted calcium carbonate due to their production process and/or storage. Such impurities may be adsorbed on some or all of the acidic and/or basic sites of the surface-reacted calcium carbonates, which may reduce its catalytic activity.

However, it is to be understood that the activation step does not affect the chemical composition of the surface-reacted calcium carbonate, i.e., no decomposition of the surface-reacted calcium carbonate, e.g., by extrusion of carbon dioxide, occurs. Therefore, the physical properties of the surface-reacted calcium carbonate and the dry surface-reacted calcium carbonate are essentially the same, i.e., the volume median particle size (d₅₀) and/or the top cut (d₉₈) value and/or the specific surface area (BET), preferably the specific surface area (BET), of the dry surface-reacted calcium carbonate differs from the corresponding property of the surface-reacted calcium carbonate by not more than 10%, preferably not more than 5%, more preferably not more than 2%.

Therefore, in a preferred embodiment, the dry surface-reacted calcium carbonate has a specific surface area of from 10 m²/g to 200 m²/g, preferably from 20 m²/g to 180 m²/g, more preferably from 25 m²/g to 160 m²/g, even more preferably from 30 m²/g to 140 m²/g, most preferably from 50 m²/g to 140 m²/g, measured using nitrogen and the BET method. For example, the dry surface-reacted calcium carbonate has a specific surface area of from 75 m²/g to 120 m²/g, measured using nitrogen and the BET method. Alternatively, the dry surface-reacted calcium carbonate has a specific surface area of from 10 m²/g to 200 m²/g, preferably from 20 m²/g to 180 m²/g, more preferably from 25 m²/g to 175 m²/g, even more preferably from 30 m²/g to 170 m²/g, most preferably from 50 m²/g to 165 m²/g, measured using nitrogen and the BET method. For example, the dry surface-reacted calcium carbonate has a specific surface area of from 75 m²/g to 165 m²/g, for example about 160 m²/g, measured using nitrogen and the BET method. The BET specific surface area in the meaning of the present invention is defined as the surface area of the particles divided by the mass of the particles. As used herein, the specific surface area is measured by adsorption using the BET isotherm (ISO 9277:2010) and is specified in m²/g.

According to a preferred embodiment of the present invention, the dry surface-reacted calcium carbonate has a specific surface area of from 75 m²/g to 165 m²/g, for example about 160 m²/g, measured using nitrogen and the BET method.

It is furthermore preferred that the dry surface-reacted calcium carbonate particles have a volume median particle size d₅₀ (vol) of from 0.5 to 50 μm, preferably from 1 to 30 μm, more preferably 1.5 to 20 μm, even more preferably from 2 to 12 μm, and most preferably from 5 to 10 μm.

It may furthermore be preferred that the dry surface-reacted calcium carbonate particles have a top cut des (vol) value of from 1 to 120 μm, preferably from 2 to 100 μm, more preferably 5 to 50 μm, even more preferably from 8 to 25 μm, and most preferably from 10 to 20 μm.

According to a preferred embodiment of the present invention, the dry surface-reacted calcium carbonate has a volume median particle size d₅₀ (vol) of from 0.5 to 50 μm, preferably from 1 to 30 μm, more preferably 1.5 to 20 μm, even more preferably from 2 to 12 μm, and most preferably from 5 to 10 μm and has a specific surface area of from 75 m²/g to 165 m²/g, for example about 160 m²/g, measured using nitrogen and the BET method.

Thus, it is preferred that the dry surface-reacted calcium carbonate of step d) has
i) a residual total moisture content from 0.01 wt.-% to 0.75 wt.-%, preferably from 0.02 wt.-% to 0.5 wt.-%, based on the total dry weight of the surface-reacted calcium carbonate, and/or ii) a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or iii) a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

In a preferred embodiment of the present invention, activation step d) is performed at a temperature from 150° C. to 400° C., more preferably from 150° C. to 300° C. Additionally or alternatively, activation step d) may be performed for a duration of at least 0.5 h, preferably of at least 1 h, and more preferably of at least 2 h, for example at least 4 h, optionally at a pressure of less than 101.3 kPa.

Activation step d) may take place using any suitable heating equipment by any method known to the skilled person and can, for example, include equipment such as an evaporator, a flash drier, an oven, preferably selected from a gravity convection oven, a mechanical convection oven, a vacuum oven and a muffle oven; a hot plate, optionally equipped with a heating block, a spray drier (such as a spray drier sold by Niro and/or Nara), and/or drying in a vacuum chamber. Additionally or alternatively, activation step d) may be performed by hot air drying, IR radiation drying or UV radiation drying.

Preferably, activation step d) is performed in an oven, such as a muffle oven or on a hot plate, optionally equipped with a heating block.

In a preferred embodiment of the present invention, the dry surface-reacted calcium carbonate has a specific surface area from 10 to 200 m²/g, preferably from 20 to 180 m²/g, more preferably from 75 m²/g to 165 m²/g measured using nitrogen and the BET method according to ISO 9277:2010, and
i) a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or ii) a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

In a particularly preferred embodiment of the present invention, the dry surface-reacted calcium carbonate has a specific surface area from 20 to 180 m²/g, preferably from 25 to 160 m²/g, more preferably from 75 m²/g to 165 m²/g measured using nitrogen and the BET method according to ISO 9277:2010, and
i) a total number of basic sites from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites 0.05 to 0.5 mmol/g, more preferably from 0.10 to 0.45 mmol/g, based on the total dry weight of the surface-reacted calcium carbonate, determined by temperature-programmed desorption with ammonia.

In a preferred embodiment of the present invention, the dry surface-reacted calcium carbonate has a specific surface area from 10 to 200 m²/g, preferably from 20 to 180 m²/g, more preferably from 75 m²/g to 165 m²/g measured using nitrogen and the BET method according to ISO 9277:2010, and is obtained in step d) by activation at a temperature from 150° C. to 400° C., more preferably from 150° C. to 300° C.

In another preferred embodiment of the present invention, the dry surface-reacted calcium carbonate has a specific surface area from 10 to 200 m²/g, preferably from 20 to 180 m²/g, more preferably from 75 m²/g to 165 m²/g measured using nitrogen and the BET method according to ISO 9277:2010, and
i) a total number of basic sites from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites 0.05 to 0.5 mmol/g, more preferably from 0.10 to 0.45 mmol/g, based on the total dry weight of the surface-reacted calcium carbonate, determined by temperature-programmed desorption with ammonia, and is obtained in step d) by activation at a temperature from 150° C. to 400° C., more preferably from 150° C. to 300° C.

### Step e)—Reacting the First and the Second Substrate

According to step e) of the process of the present invention, the first substrate of step a) and the second substrate of step b) are reacted in the presence of the dry surface-reacted calcium carbonate of step d) to obtain a reaction mixture comprising one or more condensation products and one or more condensation byproducts.

During step e), the first substrate and the second substrate react with each other in a condensation reaction to form one or more condensation products and one or more condensation byproducts. The number and relative amount of condensation products will depend on the different constitutional isomers (e.g., regioisomers), diastereomers (e.g., (E)- and (Z)-alkenes) and enantiomers (e.g., (R)- and (S)-isomers), which may be formed due to the presence of several reactive sites within the first substrate and/or the second substrate, condensation products originating from the self-reaction of one or two molecules of the first substrate and/or second substrate, and polycondensation reactions, which may occur, if the one or more condensation product represents a compound having a C═O group and/or an activated hydrogen.

For the purposes of the present invention, a “polycondensation reaction” is a sequential condensation reaction, wherein at least 3, preferably at least 4, more preferably at least 10 individual molecules of the first substrate and/or second substrate are bonded with one another. However, it is preferred that polycondensation does not occur or only occurs to a small extent, e.g., such that less than 5 wt.-%, preferably less than 2 wt.-%, more preferably less than 1 wt.-% of the one or more condensation products are polycondensation products. The skilled person knows how to avoid the formation of large amounts of polycondensation products, e.g., by selecting appropriate relative amounts of the first and the second substrate and/or by stopping the reaction at a low conversion of the first substrate, e.g., at 10 mol-% conversion, preferably at 30 mol-% conversion, or at 50 mol-% conversion.

In the cases, where the first substrate is a compound according to formula (1), wherein X═H or X═R^{X}, the at least one condensation byproduct is water. In the cases, where the first substrate is a compound according to formula (1), wherein X is a leaving group LG, the one or more condensation byproduct comprises said leaving group LG and a hydrogen atom, i.e., if the leaving group LG is a halide group, an acyloxy group, a sulfate group or a sulfite group, the at least one condensation byproduct is a hydrogen halide, a carboxylic acid, a hydrosulfate or a hydrosulfite, respectively. Thus, in a preferred embodiment of the present invention, the condensation byproduct is water.

It is appreciated that the composition of the reaction mixture comprising one or more condensation products and one or more condensation byproducts obtained in step d) depends on the selection of the first substrate provided in step a) and the second substrate in step b).

In a preferred embodiment of the present invention, a first substrate being a compound according to formula (1) is reacted with a second substrate being a compound according to formula (2) to obtain a reaction mixture comprising the one or more condensation products and the one or more condensation byproducts.

In a preferred embodiment of the present invention, a first substrate being a compound according to formula (1) is reacted with a second substrate being a compound according to formula (2), wherein X═H or R^{X}. In said embodiment, the reaction generally proceeds according to the following scheme (A):

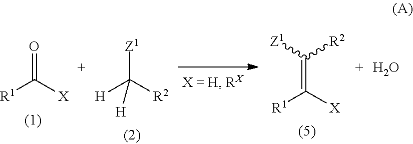

In scheme (A), the waved lines indicate that the obtained one or more condensation product is a compound according to formula (5) and may be present as its (E)-isomer, its (Z)-isomer, or a mixture thereof. The one or more condensation byproduct is water.

In one embodiment of the present invention, the first substrate is a compound according to formula (1) and the second substrate is a compound according to formula (2), and wherein
R¹ is a hydrogen atom or an organyl group R¹¹,
X is a hydrogen atom or an organyl group R^{X},
R² is a hydrogen atom or an organyl group R²¹, and
Z¹ is an electron-withdrawing group selected from the group consisting of an acyl group, a formyl group, an acetyl group and a nitro group.

In said embodiment, the one or more condensation product obtained in reaction step e) is a compound according to formula (5), wherein R¹═H or R¹¹, X═H or R^{X}, R²═H or R²¹ and Z¹ is an acyl group, a formyl group, an acetyl group or a nitro group.

Thus, the first substrate and the second substrate may react with one another in an aldol condensation reaction. For the purposes of the present invention, the aldol condensation reaction is defined as a reaction according to scheme (A), wherein R¹═H or R¹¹, X═H or R^{X}, preferably H, Z¹ is selected from the group consisting of an acyl group, a formyl group and an acetyl group, and R²═H or R²¹.

In one specific embodiment, the first substrate and the second substrate may react with one another in a Claisen-Schmidt reaction. For the purposes of the present invention, the Claisen-Schmidt reaction is defined as a reaction according to scheme (A), wherein R¹═R¹¹, preferably an aryl group, X═H, R²═H or R²¹ and Z¹ is selected from the group consisting of an acyl group, a formyl group and an acetyl group, wherein the compound according to formula (1) does not comprise a hydrogen atom in the α-position of the carbonyl group.

Alternatively, the first substrate and the second substrate may react with one another in an Henry reaction. For the purposes of the present invention, the Henry reaction is defined as a reaction according to scheme (A), wherein R¹═H or R¹¹, X═H or R^{X}, preferably H, Z¹ is a nitro group, and R² ═H or R²¹, preferably H.

In a particularly preferred embodiment of the present invention, the first substrate is a compound according to formula (1) and the second substrate is a compound according to formula (2), and wherein
R¹ is a hydrogen atom or an organyl group R¹¹,
X is a hydrogen atom,
R² is a hydrogen atom or an organyl group R²¹, and
Z¹ is selected from the group consisting of an acyl group, a formyl group, an acetyl group, a nitro group and a nitrile group.

In said embodiment, the first substrate is an aldehyde, and the one or more condensation product obtained in reaction step e) is a compound according to formula (5), wherein R¹═H or R¹¹, X ═H, R²═H or R²¹ and Z¹ is an acyl group, a formyl group, an acetyl group, a nitro group or a nitrile group.

Thus, in a particularly preferred embodiment of the present invention, the first substrate and the second substrate may react with one another in an aldol condensation reaction, wherein the first substrate is an aldehyde and the second substrate is an aldehyde or a ketone, more precisely, wherein R¹═H or R¹¹, X═H, Z¹ is selected from the group consisting of an acyl group, a formyl group and an acetyl group, and R²═H or R²¹.

In another preferred embodiment of the present invention, the first substrate and the second substrate may react with one another in an Henry reaction, wherein the first substrate is an aldehyde and the second substrate is a nitroalkane derivative, more precisely, wherein R¹═H or R¹¹, X═H, Z¹ is a nitro group, and R²═H or R²¹, preferably H.

In another embodiment of the present invention, the first substrate is a compound according to formula (1) and the second substrate is a compound according to formula (2), and wherein
R¹ is a hydrogen atom or an organyl group R¹¹,
X is a hydrogen atom or an organyl group R^{X},
R² is an electron-withdrawing group Z²,
and wherein Z¹ and Z² are independently from each other selected from the group consisting of an acyl group, a formyl group, a nitro group, a nitrile group, and an acyloxy group, preferably Z¹ and Z² are the same group.

In said embodiment, the one or more condensation product obtained in reaction step e) is a compound according to formula (5), wherein R¹═H or R¹¹, X═H or R^{X}, R²═Z², and wherein Z¹ and Z² are as defined above.

Thus, the first substrate and the second substrate may react with one another in a Knoevenagel reaction. For the purposes of the present invention, the Knoevenagel reaction is defined as a reaction according to scheme (A), wherein R¹═H or R¹¹, X═H or R^{X}, preferably H, Z¹ is selected from the group consisting of an acyl group, a formyl group, a nitro group, a nitrile group, and an acyloxy group, and R²═Z², wherein Z² is selected from the group consisting of an acyl group, a formyl group, a nitro group, a nitrile group, and an acyloxy group.

In a preferred embodiment of the present invention, the first substrate is a compound according to formula (1) and the second substrate is a compound according to formula (2), and wherein
R¹ is a hydrogen atom or an organyl group R¹¹,
X is a hydrogen atom,
R² is an electron-withdrawing group Z²,
and wherein Z¹ and Z² are independently from each other selected from the consisting of an acyl group, a formyl group, a nitro group, a nitrile group, and an acyloxy group, preferably Z¹ and Z² are the same group.

In said embodiment, the one or more condensation product obtained in reaction step e) is a compound according to formula (5), wherein R¹═H or R¹¹, X═H, R²═Z², and wherein Z¹ and Z² are as defined above.

In a particularly preferred embodiment of the present invention, the first substrate is formaldehyde (i.e., a compound according to formula (1), wherein R¹═X═H), and the second substrate is a compound according to formula (2), wherein R²═Z², and wherein Z¹ and Z² are independently from each other selected from the consisting of an acyl group, a formyl group, a nitro group, a nitrile group, and an acyloxy group, preferably Z¹ and Z² are the same group.

In another embodiment of the present invention, a first substrate being a compound according to formula (1) is reacted with a second substrate being a compound according to formula (2), wherein X=LG. In said embodiment, the reaction generally proceeds according to the following scheme (B):

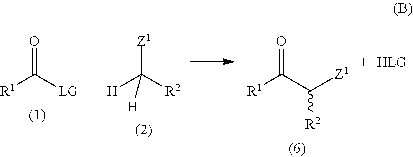

In scheme (B), the waved line indicates that the obtained one or more condensation product is a compound according to formula (6) and may be present as its (R)-isomer, its (S)-isomer, or a mixture thereof, preferably a 1:1-mixture of the (R)-isomer and the (S)-isomer, also termed racemate. The one or more condensation byproducts comprise the leaving group LG and a hydrogen atom (“HLG”).

In a preferred embodiment of the present invention, the first substrate is a compound according to formula (1) and the second substrate is a compound according to formula (2), and wherein R¹═R¹¹, X=LG, Z¹ is selected from the group consisting of an acyl group, a formyl group, an acetyl group, a nitro group, a nitrile group, an ester group, a carboxyl group, a sulfonate group, a sulfinate group, a sulfonyl group, a sulfinyl group and an isocyanate group, and R²═Z².

In another preferred embodiment of the present invention, the first substrate and the second substrate are the same compound, that is, a single compound is reacted in step e) to obtain a reaction mixture comprising one or more condensation products and one or more condensation byproducts. The reaction may take place in an intramolecular fashion and/or an intermolecular fashion.

In one embodiment of the present invention, a first substrate being a compound according to formula (3) reacts in an intramolecular fashion, wherein X═H or R^{X}. In said embodiment, the reaction generally proceeds according to the following scheme (C):

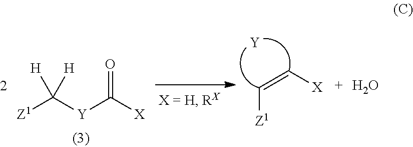

In one embodiment of the present invention, the first substrate is a compound according to formula (3), wherein Y is as defined hereinabove, X═H or R^{X}, preferably H, and Z¹ is selected from the group consisting of an acyl group, a formyl group, an acetyl group and a nitro group.

In another embodiment of the present invention, a first substrate being a compound according to formula (4) reacts in an intramolecular fashion. In said embodiment, the reaction generally proceeds according to the following scheme (D):

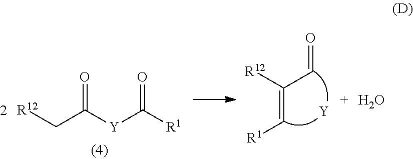

Thus, the first substrate and the second substrate may react with one another in an intramolecular aldol condensation reaction. In this embodiment, R¹², R¹, and Y are as defined above, and preferably Y is selected from the group consisting of a propylene group (CH₂—CH₂—CH₂), an ethyleneoxy group (CH₂CH₂—O), a butylene group, a propyleneoxy group, a pentylene group, a butyleneoxy group, a CH₂—C(CH₃)₂-CH₂ group and a CH₂—C(OC(═O)R^{Y6})₂-CH₂ group, wherein R^{Y6} is methyl or ethyl.

In one embodiment of the present invention, a first substrate being a compound according to formula (1), wherein R¹═—(CH₂)—R¹², reacts in an intermolecular fashion, wherein X═H or R^{X}. In said embodiment, the reaction generally proceeds according to the following scheme (E):

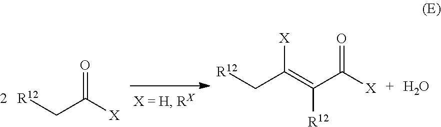

In an especially preferred embodiment of the present invention, the first substrate being a compound according to formula (1), wherein R¹═—(CH₂)—R¹², reacts in an intermolecular fashion, wherein X═H. Thus, in an exemplary embodiment of the present invention, the first substrate is acetaldehyde and the one or more condensation product is crotonaldehyde, or the first substrate is butyric aldehyde and the one or more condensation product is 2-ethylhex-2-enal, or the first substrate is phenylacetaldehyde and the one or more condensation product is 2,4-diphenylbut-2-enal.

Reaction step e) may be performed in the absence of a solvent or in the presence of a solvent.

If the reaction is performed in the presence of a solvent, the solvent is preferably selected from the group comprising acetonitrile, benzene, 1-butanol, 2-butanol, tert-butanol, chlorobenzene, chloroform, cyclohexane, 1,2-dichloroethane, diethylene glycol dimethyl ether, 1,2-dimethoxyethane, dimethyl carbonate, dimethyl formamide, dimethyl sulfoxide, 1,4-dioxane, ethanol, ethyl acetate, ethylene glycol, methanol, methyl tert-butyl ether, cyclopropyl methyl ether, N-methyl pyrrolidinone, 1-propanol, 2-propanol, propylene carbonate, tetrahydrofuran, 2-methyltetrahydrofuran, toluene, xylene, mesitylene and mixtures thereof. Further solvents, which may be used in reaction step e), include those listed in the updated and expanded GSK Solvent Sustainability Guide (C. M. Alder et al., “Updating and Expanding GSK's Solvent Sustainability Guide”, Green Chemistry 2016, 18, 3879-3890). Thereof, solvents labeled with “few known issues” or “some known issues”, and in particular solvents labeled with “few known issues” are preferred.

In a preferred embodiment of the present invention, reaction step e) is performed in the absence of a solvent.

Additionally or alternatively, it is preferred that step e) is performed in the liquid phase at a reaction temperature in the range from 20° C. to 250° C., preferably from 50° C. to 200° C., more preferably from 100 to 150° C. Preferably, step e) is performed in the liquid phase at a reaction temperature closely below or at the normal boiling point of the first substrate and/or the second substrate and/or the solvent, preferably the solvent. If the reaction is performed at a temperature above the normal boiling point of the first substrate and/or the second substrate and/or the solvent, it is preferred that the reaction is performed in a closed container under autogenous pressure and/or under reflux. While it is more convenient to carry out the inventive method under ambient pressure, increased pressure may allow for higher reaction temperatures and higher reaction rates.

For the purposes of the present invention, the normal boiling point of a substance refers to the boiling point of the essentially pure substance at standard pressure of 101.325 kPa, wherein essentially pure means that the substance comprises less than 5 wt.-%, preferably less than 2 wt.-%, more preferably less than 1 wt.-% impurities.

In still another embodiment according to the present invention, step e) is carried out under an inert gas atmosphere, wherein preferred inert gases are nitrogen, argon and mixtures thereof.

In a preferred embodiment of step e) of the present invention, the dry surface-reacted calcium carbonate is added in an amount from 0.5 to 50 wt.-%, preferably from 1 to 30 wt.-%, more preferably from 5 to 25 wt.-%, and most preferably from 10 to 20 wt.-%, based on the total weight of the first substrate.

Thus, in a particularly preferred embodiment of the present invention, the dry surface-reacted calcium carbonate is added in an amount from 0.5 to 50 wt.-%, preferably from 1 to 30 wt.-%, more preferably from 5 to 25 wt.-%, and most preferably from 10 to 20 wt.-%, based on the total weight of the first substrate, wherein the dry surface-reacted calcium carbonate has a specific surface area from 10 to 200 m²/g, preferably from 20 to 180 m²/g, more preferably from 75 m²/g to 165 m²/g measured using nitrogen and the BET method according to ISO 9277:2010, and optionally
i) a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

In another preferred embodiment of the present invention, the dry surface-reacted calcium carbonate is added in an amount from 5 to 25 wt.-%, preferably from 10 to 20 wt.-%, based on the total weight of the first substrate, wherein the dry surface-reacted calcium carbonate has a specific surface area from 10 to 200 m²/g, preferably from 20 to 180 m²/g, more preferably from 75 m²/g to 165 m²/g measured using nitrogen and the BET method according to ISO 9277:2010, and optionally
i) a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

In yet another preferred embodiment of the present invention, the dry surface-reacted calcium carbonate is added in an amount from 5 to 25 wt.-%, preferably from 10 to 20 wt.-%, based on the total weight of the first substrate, wherein the dry surface-reacted calcium carbonate has a specific surface area from 20 to 180 m²/g, preferably from 75 m²/g to 165 m²/g, measured using nitrogen and the BET method according to ISO 9277:2010, and optionally
i) a total number of basic sites from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites from 0.05 to 0.5 mmol/g, more preferably from 0.10 to 0.45 mmol/g, based on the total dry weight of the surface-reacted calcium carbonate, determined by temperature-programmed desorption with ammonia.

In still another preferred embodiment of the present invention, the dry surface-reacted calcium carbonate is added in an amount from 5 to 25 wt.-%, preferably from 10 to 20 wt.-%, based on the total weight of the first substrate, wherein the dry surface-reacted calcium carbonate has a specific surface area from 20 to 180 m²/g, preferably from 75 m²/g to 165 m²/g, measured using nitrogen and the BET method according to ISO 9277:2010, and optionally
i) a total number of basic sites from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
ii) a total number of acidic sites from 0.05 to 0.5 mmol/g, more preferably from 0.10 to 0.45 mmol/g, based on the total dry weight of the surface-reacted calcium carbonate, determined by temperature-programmed desorption with ammonia, and the first substrate is a compound according to formula (1) and the second substrate is a compound according to formula (2), and wherein
R¹ is a hydrogen atom or an organyl group R¹¹,
X is a hydrogen atom,
R² is a hydrogen atom or an organyl group R²¹, and
Z¹ is selected from the group consisting of an acyl group, a formyl group, an acetyl group and a nitro group.

Additionally or alternatively, the first substrate and the second substrate are added in step e) in a molar ratio from 1:1 to 1:20, preferably from 1:2.5 to 1:15, and more preferably from 1:5 to 1:15. An excess amount of the second substrate may increase the overall conversion of the first substrate to the desired product and may promote the reaction between the first substrate and the second substrate, if the first substrate can undergo a reaction with itself. For example, an excess of the second substrate may promote the cross-aldol reaction over the self-aldol reaction. As an illustrative example, if the first substrate is butyraldehyde and the second substrate is acetone, butyraldehyde may undergo a self-aldol condensation to 2-ethylhexenal or a cross-aldol condensation to hept-3-en-2-on. Increasing the amount of acetone relative to butyraldehyde increases the relative amount of hept-3-en-2-on over 2-ethylhexenal.

Furthermore, the reaction mixture may comprise further additives, preferably selected from the group consisting of further substrates, co-catalysts, promoters, activators, and support materials. In one embodiment, a further substrate comprising a C═O double bond and/or an activated hydrogen may be added to perform a three-component reaction. In another embodiment, the surface-reacted calcium carbonate may be fixed onto a support material, such as activated carbon, alumina or silica. In yet another embodiment, a promoter may be added in order to avoid deactivation of the catalyst, e.g., by preventing or retarding sintering of the surface-reacted calcium carbonate and/or the support material.

It is appreciated that in reaction step e), the first substrate provided in step a), the second substrate provided in step b), the surface-reacted calcium carbonate provided in step c) and optionally the solvent and/or the further additives are contacted with one another in any order.

Reaction step e) can be performed by any means known to the skilled person. For example, the reaction step e) can be carried out, e.g., in a discontinuous process, such as in a batch reactor composed of a storage tank and an agitator, e.g., a round-bottom flask; a pressure reactor, e.g., a standard glass pressure reactor, a Fisher-Porter tube, a metal pressure reactor or a microwave synthesizer. The use of pressure reactors is especially preferred, if the reaction is performed at a reaction temperature above the boiling point of the solvent and/or the first substrate and/or second substrate.

Optionally, the one or more condensation byproducts of step e) may be continuously removed from the reaction mixture during reaction step e), preferably by azeotropic distillation or reactive distillation.

Alternatively, reaction step e) can be carried out in a continuous process, e.g., in a continuous stirred-tank reactor, a semi-batch reactor, a laminar flow reactor, a microreactor, or a continuous oscillatory baffled reactor.

In yet another embodiment of the present invention, the surface-reacted calcium carbonate is provided as a bed in a heterogeneous catalytic reactor, preferably a fixed bed reactor, a trickle-bed reactor, a moving bed reactor, a rotating bed reactor, a fluidized bed reactor, a slurry reactor or an ebullated bed reactor.

Optionally, the inventive process comprises a step f) of separating the reaction mixture. The reaction mixture may be separated by any means known to the skilled person. For example, the surface-reacted calcium carbonate may be removed from the reaction mixture by solid-liquid separation, e.g., by filtration, gravity settling, centrifugation, decantation, cycloning or classifying. The surface-reacted calcium carbonate may be further purified, e.g., by washing with a solvent, such as methanol or diethyl ether, optionally re-activated in a process step according to activation step d), and re-used in reaction step e). The condensation products may be separated from the reaction mixture by one of, preferably a combination of solvent extraction, evaporation, distillation, fractional distillation and/or chromatography, such as column chromatography and high-performance liquid chromatography.

### The Inventive Use

A second aspect of the present invention relates to the use of a dry surface-reacted calcium carbonate as a catalyst, wherein the surface-reacted calcium carbonate is a reaction product of ground natural calcium carbonate-containing mineral (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, wherein the surface-reacted calcium carbonate has a specific surface area of at least 10 m²/g, measured using nitrogen and the BET method according to ISO 9277:2010, and wherein the surface-reacted calcium carbonate has been dried by heating at a temperature in the range from 100 to 500° C.

It is appreciated that the dry surface-reacted calcium carbonate is as defined hereinabove and may be obtained by a process as outlined hereinabove.

In a preferred embodiment of the present invention, the dry surface-reacted calcium carbonate has
i) a volume median particle size (d₅₀) from 0.5 to 50 μm, preferably from 1 to 30 μm, more preferably from 1.5 to 20 μm, and most preferably from 2 to 12 μm, and/or
ii) a top cut (d₉₈) value from 1 to 120 μm, preferably from 2 to 100 μm, more preferably from 5 to 50 μm, and most preferably from 8 to 25 μm, and/or
iii) a specific surface area (BET) from 10 to 200 m²/g, preferably of from 20 to 180 m²/g, more preferably from 25 to 160 m²/g, and most preferably from 30 to 140 m²/g, as measured by the BET method, and/or
iv) a residual total moisture content from 0.01 wt.-% to 0.75 wt.-%, preferably from 0.02 wt.-% to 0.5 wt.-%, based on the total dry weight of the surface-reacted calcium carbonate, and/or
v) a total number of basic sites from 0.01 to 0.6 mmol/g, preferably from 0.05 to 0.5 mmol/g, more preferably from 0.10 to 0.45 mmol/g, determined by temperature-programmed desorption with ammonia, and/or
vi) a total number of acidic sites from 0.01 to 0.6 mmol/g, preferably from 0.05 to 0.5 mmol/g, more preferably from 0.10 to 0.45 mmol/g, determined by temperature-programmed desorption with carbon dioxide.

In another preferred embodiment of the present invention, the dry surface-reacted calcium carbonate has
i) a volume median particle size (d₅₀) from 0.5 to 50 μm, preferably from 1 to 30 μm, more preferably from 1.5 to 20 μm, and most preferably from 2 to 12 μm, and/or
ii) a top cut (d₉₈) value from 1 to 120 μm, preferably from 2 to 100 μm, more preferably from 5 to 50 μm, and most preferably from 8 to 25 μm, and/or
iii) a specific surface area (BET) from 10 to 200 m²/g, preferably of from 20 to 180 m²/g, more preferably from 75 to 165 m²/g, as measured by the BET method, and/or
iv) a residual total moisture content from 0.01 wt.-% to 0.75 wt.-%, preferably from 0.02 wt.-% to 0.5 wt.-%, based on the total dry weight of the surface-reacted calcium carbonate, and/or
v) a total number of basic sites from 0.01 to 0.6 mmol/g, preferably from 0.05 to 0.5 mmol/g, more preferably from 0.10 to 0.45 mmol/g, determined by temperature-programmed desorption with ammonia, and/or
vi) a total number of acidic sites from 0.01 to 0.6 mmol/g, preferably from 0.05 to 0.5 mmol/g, more preferably from 0.10 to 0.45 mmol/g, determined by temperature-programmed desorption with carbon dioxide.

A particularly preferred embodiment of the present aspect of the invention refers to the use of surface-reacted calcium carbonate as a catalyst for a condensation reaction. Preferably, the condensation reaction refers to a reaction of a first substrate and a second substrate as defined hereinabove. Thus, it is appreciated that the condensation reaction may be an aldol condensation, a Knoevenagel condensation, an Henry reaction, or a Claisen-Schmidt condensation.

It is appreciated that the use of the dry surface-reacted calcium carbonate as a catalyst in a condensation reaction may improve the yields and/or the turnover number and/or the turnover frequency of the condensation reaction and/or improve the purity of the product of the condensation reaction, compared to the use of a soluble catalyst, such as sulfuric acid or sodium hydroxide.

The following examples are intended to further illustrate the present invention. However, these examples should not be construed as limiting the scope of the present invention in any way.

### EXAMPLES

Measurement Methods

In the following, measurement methods implemented in the examples are described.

Particle Size Distribution

Volume determined median particle size d₅₀(vol) and the volume determined top cut particle size des(vol) was evaluated using a Malvern Mastersizer 3000 Laser Diffraction System (Malvern Instruments Plc., Great Britain). The d₅₀(vol) or des(vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The sample was measured in dry condition without any prior treatment.

The weight determined median particle size d₅₀(wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high-speed stirrer and supersonicated.

Specific Surface Area (SSA)

The specific surface area was measured via the BET method according to ISO 9277:2010 using nitrogen, following conditioning of the sample by heating at 250° C. for a period of 30 minutes.

Prior to such measurements, the sample was filtered within a Buchner funnel, rinsed with deionized water and dried at 110° C. in an oven for at least 12 hours.

Intra-Particle Intruded Specific Pore Volume (in Cm³/g)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step was 20 seconds. The sample material was sealed in a 5 cm³ chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p1753-1764).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Scanning Electron Microscopy (SEM)

The samples were prepared by diluting 50 to 150 μl slurry samples with 5 ml water. The amount of slurry sample depends on solids content, mean value of the particle size and particle size distribution. The diluted samples were filtrated by using a 0.8 μm membrane filter. A finer filter was used when the filtrate is turbid. A doubled-sided conductive adhesive tape was mounted on a SEM stub. This SEM stub was then slightly pressed in the still wet filter cake on the filter. The SEM stub was then sputtered with 8 nm Au. Subsequently, the prepared samples were examined by: a Sigma VP field emission scanning electron microscope (FESEM) (Carl Zeiss AG, Germany) and a variable pressure secondary electron detector (VPSE) and/or secondary electron detector (SE) with a chamber pressure of about 50 Pa. The investigation under the FESEM (Zeiss Sigma VP) was done at 5 kV (Au).

X-Ray Diffraction (XRD), X-Ray Photoelectron Spectroscopy (XPS), Thermogravimetric Analysis (TGA)

XRD patterns were recorded using a Bruker D2 Phaser powder X-ray diffractometer using Co radiation source, CoKa=1.789 Å. Measurements were carried out between 10-70° 2θ using a scan speed of 0.5 s per step. TGA was conducted using a Mettler Toledo TGA/DSC 3⁺. The samples were heated from 25 up to 600° C. with a ramp of 25° C. and a 10 min hold at 105° C. and 500° C., with an air flow of 80 ml/min. XPS experiments were carried out in a Kratos AXIS Ultra DLD spectrometer using a monochromatic A1 Kα radiation (hu=1486.6 eV) operating at 225 W (15 mA, 15 kV). Instrument base pressure was 5×10⁻¹⁰ Torr.

Further Experimental Techniques

The Ca and P contents of the SRCC solids were prepared by dissolving a sample of the SRCC in aqua regia (a mixture of 1 part per volume of nitric acid (70 wt.-% in water) and 3 parts per volume of hydrochloric acid (35 wt.-% in water)), diluting the obtained solution with water until an about four-fold increase in volume, and analyzing the diluted solution via the inductively coupled plasma optical emission spectroscopy (ICP-OES) technique using a Perkin Elmer Avio 500 device. The Ca and P contents were determined using a calibration curve. The carbon content of the SRCC was obtained from EA and was carried out using a Fisons NA1500 NCS analyser. Infrared spectra were recorded using a Perkin Elmer Spectrum Two FT-IR spectrometer.

Adsorbed Ammonia and Adsorbed Carbon Dioxide Temperature Programmed Desorption (NH₃-TPD and CO₂-TPD)

The measurements were performed using a Micromeritics ASAP2920 apparatus. 0.1 g of sample was dried in situ under an He flow with a temperature ramp of 5° C. min⁻¹ up to 400° C.

For the NH₃-TPD measurements, the sample was cooled to 100° C. At this point, 20 pulses of 5 cm³ 10 vol.-% NH₃ in He were dosed over the sample (corresponding to an NH₃ flow of 25.3 cm³ min⁻¹). The sample was then heated to 600° C. with a ramp of 5° C. min⁻¹ to induce desorption of NH₃. The amount of NH₃ desorbed over time was determined using a thermal conductivity detector (TCD). The TCD concentration was plotted over time for the quantitative evaluation and over temperature to determine the temperature position of the desorption peaks. In both cases, a peak deconvolution was performed. To obtain the total amount of desorbed NH₃, a baseline subtraction and full integration of the desorption feature has been performed. Peak deconvolution was performed using the software Fityk.

After obtaining the area under the curve (AUC, A) (from Fityk), the AUC is converted into a quantifiable amount of NH₃ (n_{NH3} in mmol/g) using the below formulae:

Aᵣ=A/100%

V_{NH3,abs}=Aᵣ·V

V_{NH3}=V_{NH3,abs}/mₛₐₘₚₗₑ

m_{NH3}=V_{NH3}·ρ_{NH3}

n_{NH3}=m_{NH3}/M_{NH3}

ρ_{NH3}=0.76 kg/m³,M_{NH3}=17 g/mol

A=obtained Area (%·min), Aᵣ=Area (min), V=Flow 25.2 (cm³/min)

V_{NH3,abs}=absolute amount of desorbed NH₃ (cm³)

V_{NH3}=amount of desorbed NH₃ per g of sample (cm³/g)

For the CO₂-TPD measurements, the sample was cooled to 50° C. and a procedure similar to the one described for NH₃-TPD was employed. The number of basic sites was determined according to the calculation above, using the values ρ_{CO2}=1.98 kg/m³ and M_{CO2}=44.01 g/mol. For calculating the number of acidic or basic sites, it was assumed that only one molecule of NH₃ or CO₂ can adsorb on a single site.

2. Materials Used

Surface-Reacted Calcium Carbonate (SRCC)

SRCC1

SRCC1 is commercially available from Omya International AG with a d₅₀(vol)=2.4 μm, a des(vol)=9 μm, and SSA=21 m²/g. The intra-particle intruded specific pore volume is 0.442 cm³/g (for the pore diameter range of 0.004 to 0.34 μm).

SRCC2

SRCC2 has d₅₀(vol)=6.6 μm, a des(vol)=13.7 μm, a SSA=56.7 m²g⁻¹ and an intra-particle intruded specific pore volume of 0.939 cm³/g (for the pore diameter range of 0.004 to 0.51 μm).

SRCC2 was obtained by preparing 350 L of an aqueous suspension of ground calcium carbonate in a mixing vessel by adjusting the solids content of a ground limestone calcium carbonate from Omya SAS, Orgon having a weight based median particle size d₅₀(wt) of 1.3 μm, as determined by sedimentation, such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

SRCC3

SRCC3 has d₅₀(vol)=5.8 μm, a d₉s(vol)=15.4 μm, a SSA=156.2 m²g⁻¹ and an intra-particle intruded specific pore volume of 1.070 cm³/g (for the pore diameter range of 0.004 to 0.34 μm).

SRCC3 was obtained by preparing 10 L of an aqueous suspension of ground calcium carbonate in a mixing vessel by adjusting the solids content of a ground marble calcium carbonate from Hustadmarmor Norway such that a solids content of 10 wt.-%, based on the total weight of the aqueous suspension, is obtained. The ground calcium carbonate had a weight based particle size distribution of 90% less than 2 μm, as determined by sedimentation. Additionally, a phosphoric acid solution was prepared such that it contained 30% phosphoric acid, based on the total weight of the solution.

Whilst mixing the slurry, 1.8 kg of the phosphoric acid solution was added over 10 minutes. After 20% of the total acid solution was added, 53 g of citric acid anhydride powder was added to the slurry. Throughout the whole experiment the temperature of the suspension was maintained at 70° C.+/−1° C. Finally, after the addition of the acid, the suspension was stirred for additional 5 minutes before removing it from the vessel and allowing it to cool.

SRCC4

SRCC4 has d₅₀(vol)=3.8 μm, a d₉s(vol)=47.2 μm, a SSA=86.7 m²g⁻¹ and an intra-particle intruded specific pore volume of 0.286 cm³/g (for the pore diameter range of 0.004 to 0.11 μm).

SRCC4 was obtained by preparing 10 L of an aqueous suspension of ground calcium carbonate in a mixing vessel by adjusting the solids content of a ground marble calcium carbonate from Karabiga, Turkey such that a solids content of 15 wt.-%, based on the total weight of the aqueous suspension, is obtained. The ground calcium carbonate had a weight based particle size distribution of 90% less than 1 μm, as determined by sedimentation. In addition, a phosphoric acid solution was prepared such that it contained 30% phosphoric acid, based on the total weight of the solution.

Whilst mixing the slurry, 1.3 kg of the phosphoric acid solution was added over 10 minutes. Throughout the whole experiment the temperature of the suspension was maintained at 70° C.+/−1° C. Finally, after the addition of the acid, the suspension was stirred for additional 5 minutes before removing it from the vessel and allowing it to cool.

SRCC5

SRCC5 has d₅₀(Vol)=8.3 μm, a d₉s(vol)=18.7 m, a SSA=105.5 m²g⁻¹ and an intra-particle intruded specific pore volume of 1.565 cm³/g (for the pore diameter range of 0.004 to 0.66 μm).

SRCC5 was obtained by preparing 10 L of an aqueous suspension of ground calcium carbonate in a mixing vessel by adjusting the solids content of a ground marble calcium carbonate from Karabiga, Turkey such that a solids content of 15 wt.-%, based on the total weight of the aqueous suspension, is obtained. The ground calcium carbonate had a weight based median particle size d₅₀(wt) of 1.4 μm, as determined by sedimentation. In addition, a phosphoric acid solution was prepared such that it contained 30% phosphoric acid, based on the total weight of the solution.

Whilst mixing the slurry, 2.8 kg of the phosphoric acid solution was added over 15 minutes. Throughout the whole experiment the temperature of the suspension was maintained at 70° C.+/−1° C. Finally, after the addition of the acid, the suspension was stirred for additional 5 minutes before removing it from the vessel and allowing it to cool.

Other Reagents

All commercial reagents were used as received without further purification. Butyraldehyde (>99.5%), malononitrile (>99%), nitromethane (>99%), mesitylene (98%) HAP-L nanopowder (<200 nm, particle size, >97%), HAP-H (5 μm particle size) and TiO₂ nanopowder (<100 nm particle size, 99.5%) were purchased from Sigma-Aldrich. Benzaldehyde (>98%), acetophenone (98%), calcium carbonate (CaCO₃) (>98%) and MgO (98%) were obtained from Acros organics.

The SRCCs were activated by heating to 200° C. for 4 h in a laboratory oven under static air. The properties of the surface-reacted calcium carbonates (labeled “before”) and the dry surface-reacted calcium carbonates (labeled “after”) are shown in Tables 1-3. The properties of several commercially available catalysts are also shown in Table 1.

TABLE 1
Nitrogen physisorption measurements for SRCC catalysts.

| | |
|---|---|
| BET surface area | Total pore volume |
| (m²/g) | (cm³/g) |

| Entry | Catalyst | Before^{a} | After^{b} | Before^{a} | After^{b} |
|---|---|---|---|---|---|
| 1 | SRCC1 | 21.0 | 19.3 | 0.032 | 0.133 |
| 2 | SRCC2 | 56.7 | 58.1 | 0.101 | 0.417 |
| 3 | SRCC3 | 156.2 | 160.3 | 0.304 | 0.865 |
| 4 | SRCC4 | 86.7 | 85.5 | 0.193 | 0.181 |
| 5 | SRCC5 | 105.5 | 106.7 | 0.673 | 0.650 |
| 6 | MgO | n.d. | 239.5 | n.d. | 0.239 |
| 7 | TiO₂ | n.d. | 7.4 | n.d. | 0.033 |
| 8 | CaCO₃ | n.d. | 6.6 | n.d. | 0.049 |
| 9 | HAP-L^{c} | 9.4 | n.d. | n.d. | n.d. |
| 10 | HAP-H^{c} | 100 | n.d. | n.d. | n.d. |

^{a}before thermal activation;
^{b}after thermal activation at 200° C. for 4 h;
^{c}commercially available information;
n.d.—not determined.

TABLE 2
Bulk and surface composition of the SRCC catalysts.

| | ICP-OES XPS | | | XPS | | |
|---|---|---|---|---|---|---|
| | | | Ca/P | | | Ca/P |
| | Ca | P | atomic | Ca | P | atomic |
| Catalyst | (wt %) | (wt %) | ratio | (% at.) | (% at.) | ratio |
| SRCC1 | 38.42 | 4.17 | 7.13 | 14.2 | 6.7 | 2.1 |
| SRCC2 | 38.29 | 7.55 | 3.93 | 13.0 | 6.7 | 1.9 |
| SRCC3 | 40.67 | 6.34 | 4.95 | 14.4 | 7.8 | 1.9 |
| SRCC4 | 38.14 | 7.59 | 3.88 | 14.1 | 6.7 | 2.1 |
| SRCC5 | 37.44 | 12.36 | 2.34 | 14.5 | 8.1 | 1.8 |

TABLE 3
Number of acidic and basic sites of the dry surface-reacted
calcium carbonates determined by NH₃ and CO₂-TPD.

| | | CO₂-TPD | NH₃-TPD |
|---|---|---|---|
| | | Total number | Total number |
| | | of basic sites | of acidic sites |
| Entry | Catalyst | (mmol/g) | (mmol/g) |
| 1 | SRCC1 | 0.05 | 0.03 |
| 2 | SRCC2 | 0.08 | 0.09 |
| 3 | SRCC3 | 0.33 | 0.19 |
| 4 | SRCC4 | 0.15 | 0.13 |
| 5 | SRCC5 | 0.08 | 0.12 |

3. Organic Condensation Reactions

Aldol Condensation

The aldol condensation of butyraldehyde was performed in a batch reaction system, under vigorous magnetic stirring and a nitrogen atmosphere. Prior to the reaction, all the SRCC solids were thermally activated at 200° C. for 4 h. In a typical experiment, a 50 mL two-necked flask connected to a reflux condenser was filled with 55.6 mmol of butyraldehyde, 3 mol % of catalyst and 36 mmol mesitylene as the internal standard. After passing the N₂ through the headspace of the reaction system and setting a constant-vigorous stirring rate, the reaction mixture was heated up to a temperature of 130° C. The progress of the reaction was monitored by taking samples from the reaction media at different intervals of time (2-22 h). The conversion of butyraldehyde was determined by ¹H NMR (CDCl₃). The ¹H spectra (400 MHz) were recorded on an Agilent MRF400 or a Varian AS400 spectrometer at 25° C. The chemical shifts are reported in the standard 5 notation of parts per million, referenced to residual peak of the solvent, as determined relative to Me₄Si (6=0 ppm).

Catalyst performance was evaluated at 130° C. for 22 h under solvent-free conditions. The results are presented in Table 4. No conversion was noted in the absence of any catalysts after 2 or 6 h, whereas a limited activity was seen at 22 h (entry 1). Entries 2 to 6 are commercially available solid base catalysts such as MgO, TiO₂, CaCO₃ and HAPs, respectively. Compared to commercially existing solid base catalysts, the SRCC catalysts SRCC3, SRCC4 and SRCC5 (entry 9-11) exhibited excellent catalytic activity. In most cases, full conversion after 22 h was achieved. A mixture of calcium carbonate and HAP catalysts was also tested (entry 12), which did not achieve the catalytic activity attained by the above mentioned SRCC catalysts.

TABLE 4
Screening of self-aldol condensation reaction of butyraldehyde
into 2-ethylhexenal using various catalysts.

| | | Catalyst | Time (h) | | | | | |
|---|---|---|---|---|---|---|---|---|
| | | amount | 2 | | 6 | | 22 | |
| Entry | Catalyst | (g) | X (%) | Y (%) | X (%) | Y (%) | X (%) | Y (%) |
| 1 | — | — | 0 | 0 | 0 | 0 | 8 | 6 |
| 2 | MgO | 0.07 | 0 | 0 | 30 | 29 | 71 | 70 |
| 3 | TiO₂ | 0.13 | 0 | 0 | 9 | 9 | 50 | 47 |
| 4 | CaCO₃ | 0.17 | 2 | 2 | 40 | 39 | 60 | 59 |
| 5 | HAP-L | 0.84 | 7 | 7 | 41 | 39 | 62 | 60 |
| 6 | HAP-H | 0.84 | 10 | 10 | 53 | 53 | 70 | 68 |
| 7 | SRCC1 | 0.30 | 0 | 0 | 28 | 28 | 50 | 48 |
| 8 | SRCC2 | 0.5 | 9 | 9 | 29 | 29 | 57 | 55 |
| 9 | SRCC3 | 0.72 | 73 | 72 | 100 | 99 | n.d. | n.d. |
| 10 | SRCC4 | 0.74 | 18 | 18 | 70 | 69 | 88 | 85 |
| 11 | SRCC5 | 0.73 | 58 | 57 | 91 | 89 | 100 | 98 |
| 12 | CaCO₃ + HAP-L | 0.72 | 3 | 3 | 10 | 10 | 43 | 43 |

Reaction conditions: Temperature = 130° C.; Catalyst loading = 3 mol %; X = Conversion, Y = Yield; HAP-L = Hydroxyapatite with lower surface area (9.4 m²/g); HAP-H = Hydroxyapatite with higher surface area (100 m²/g); Entry 12, catalyst loading - CaCO₃ = 0.5 mol %, HAP-L = 2.5 mol %; Conversion and Yield were determined by ¹H NMR (CDCl₃) using mesitylene as an internal standard.

Other Condensation Reactions

To further demonstrate the scope of SRCC catalysts used in C—C coupling reactions, the most active catalyst SRCC3 was tested in other prototypical condensation reactions. The reaction conditions were optimized similar to the self-aldol condensation reactions and the best results are displayed in the Table 5.

TABLE 5

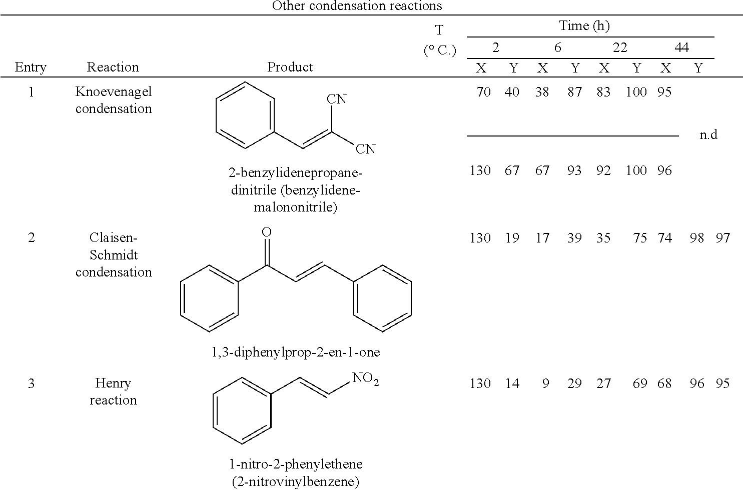

Reaction conditions: Catalyst Loading = 3 mol %; Catalyst: SRCC3; T = Temperature; X = Conversion (%), Y = Yield (%); n.d. = not determined; Entry 1, substrate 1 = benzaldehyde, substrate 2 = malononitrile; Entry 2, substrate 1 = benzaldehyde, substrate 2 = acetophenone; Entry 3, substrate 1 = benzaldehyde, substrate 2 = nitromethane; Substrate ratio: 1:2; Conversion and Yield were determined by ¹H NMR (CDCl₃) using mesitylene as an internal standard.

1. A method for performing a condensation reaction by heterogeneous catalysis, the method comprising the steps of
a) providing a first substrate comprising a C═O double bond;
b) providing a second substrate comprising an activated hydrogen;
c) providing a surface-reacted calcium carbonate,
wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source,
and wherein the surface-reacted calcium carbonate has a specific surface area of at least 10 m²/g, measured using nitrogen and the BET method according to ISO 9277:2010;
d) activating the surface-reacted calcium carbonate of step c) at a temperature in the range from 100 to 500° C. to obtain a dry surface-reacted calcium carbonate;
e) reacting the first substrate of step a) and the second substrate of step b) in the presence of the dry surface-reacted calcium carbonate of step d) to obtain a reaction mixture comprising one or more condensation products and one or more condensation byproducts.

2. The method of claim 1, wherein the first substrate is a compound 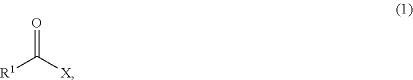
according to formula (1)
wherein R¹ is selected from the group consisting of
i) a hydrogen atom, and
ii) an organyl group R¹¹, wherein R¹¹ is optionally substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group;
and wherein X is selected from the group consisting of
i) a hydrogen atom,
ii) an organyl group R^{X}, wherein R^{X} is optionally substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group, and
iii) a leaving group LG.

3. The method of claim 1, wherein
the second substrate is a compound according to formula (2) 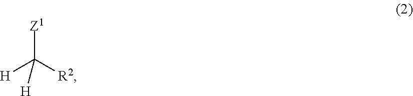
wherein Z¹ is an electron-withdrawing group,
and wherein R² is selected from the group consisting of
i) a hydrogen atom,
ii) an organyl group R²¹, wherein R²¹ is optionally substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group, and
iii) an electron-withdrawing group Z²,
with the proviso that, if Z¹ is an electron-withdrawing group other than an acyl group, a formyl group, an acetyl group or a nitro group, then R² is an electron-withdrawing group Z².

4. The method of claim 1, wherein the first substrate is a compound according to formula (1) 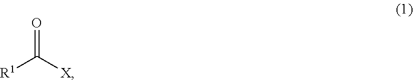
and the second substrate is a compound according to formula (2), 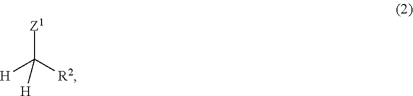
and wherein
R¹ is a hydrogen atom or an organyl group R¹¹, wherein R¹¹ is optionally substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group,
X is a hydrogen atom,
R² is a hydrogen atom or an organyl group R²¹, wherein R²¹ is optionally substituted by one or more groups selected from the group consisting of a halide group, a hydroxy group, an oxo group, an alkyl group, a vinyl group, an alkoxy group, an aryloxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group, and
Z¹ is an electron-withdrawing group selected from the group consisting of an acyl group, a formyl group, an acetyl group and a nitro group.

5. The method of claim 1, wherein the first substrate is a compound according to formula (1) 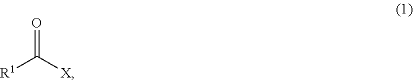
and the second substrate is a compound according to formula (2), 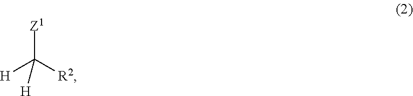
and wherein
R¹ is a hydrogen atom or an organyl group R¹¹, wherein R¹¹ is optionally substituted by one or more groups selected from the group consisting of a halide group, a hydroxy group, an oxo group, an alkyl group, a vinyl group, an alkoxy group, an acyloxy group, a carboxyl group, an epoxy group, an anhydride group, an ester group, an aldehyde group, an amino group, a ureido group, an azide group, a phosphonate group, a phosphine group, a sulfonate group, a sulfinate group, a sulfonyl group, a sulfinyl group, a sulfide group or disulfide group, an isocyanate group or masked isocyanate group, a thiol group, a nitrile group, an amine group, a phenyl group, a benzyl group, a styryl group and a benzoyl group,
X is a hydrogen atom,
Z¹ is an electron-withdrawing group,
R² is an electron-withdrawing group Z²,
and wherein Z¹ and Z² are independently from each other selected from the group consisting of an acyl group, a formyl group, a nitro group, a nitrile group, and an ester group.

6. The method of claim 1, wherein the first substrate and the second substrate are the same compound.

7. The method of claim 1, wherein the surface-reacted calcium carbonate of step c) has
i) a volume median particle size (d₅₀) from 0.5 to 50 μm, and/or
ii) a top cut (d₉₈) value from 1 to 120 μm, and/or
iii) a specific surface area (BET) from 10 to 200 m²/g, as measured by the BET method.

8. The method of claim 1, wherein
the dry surface-reacted calcium carbonate of step d) has
i) a residual total moisture content from 0.01 wt.-% to 0.75 wt.-%, 0.02 and/or
ii) a total number of basic sites from 0.01 to 0.6 mmol/g, based on the total dry weight of the surface-reacted calcium carbonate, determined by temperature-programmed desorption with ammonia, and/or
iii) a total number of acidic sites from 0.01 to 0.6 mmol/g, based on the total dry weight of the surface-reacted calcium carbonate, determined by temperature-programmed desorption with carbon dioxide.

9. The method of claim 1, wherein
the one or more H₃G⁺ ion donors are selected from the group consisting of hydrochloric acid, sulfuric acid, sulfurous acid, phosphoric acid, citric acid, oxalic acid, an acidic salt, acetic acid, formic acid, and mixtures thereof.

10. The method of claim 1, wherein activation step d) is performed at a temperature from 150° C. to 400° C., and/or for a duration of at least 0.5 h, optionally at a pressure of less than 101.3 kPa.

11. The method of claim 1, wherein reaction step e) is performed
i) in the absence of a solvent or in the presence of a solvent, and/or
ii) in the liquid phase at a reaction temperature in the range from 20° C. to 250° C.

12. The method of claim 1, wherein in reaction step e)
i) the dry surface-reacted calcium carbonate is added in an amount from 0.5 to 50 wt., based on the total weight of the first substrate, and/or
ii) the first substrate and the second substrate are added in a molar ratio from 1:1 to 1:20.

13. A catalyst comprising a dry surface-reacted calcium carbonate,
wherein the surface-reacted calcium carbonate is a reaction product of ground natural calcium carbonate-containing mineral (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and
wherein the surface-reacted calcium carbonate has a specific surface area of at least 10 m²/g, measured using nitrogen and the BET method according to ISO 9277:2010, and
wherein the surface-reacted calcium carbonate has been dried by heating at a temperature in the range from 100 to 500° C.

14. The catalyst of claim 13, wherein the dry surface-reacted calcium carbonate has
i) a volume median particle size (d₅₀) from 0.5 to 50 μm, and/or
ii) a top cut (d₉₈) value from 1 to 120 μm, and/or
iii) a specific surface area (BET) from 10 to 200 m²/g, as measured by the BET method, and/or
iv) a residual total moisture content from 0.01 wt.-% to 0.75 wt.-%, based on the total dry weight of the surface-reacted calcium carbonate, and/or
v) a total number of basic sites from 0.01 to 0.6 mmol/g, determined by temperature-programmed desorption with ammonia, and/or
vi) a total number of acidic sites from 0.01 to 0.6 mmol/g, determined by temperature-programmed desorption with carbon dioxide.

15. (canceled)
